(19) United States
(12) Patent Application Publication
(10) Pub. No.: US 2025/0295246 A1
(43) Pub. Date: Sep. 25, 2025

(54) DETECTING PRESENCE OR ABSENCE OF BABIES IN CRADLES USING MONITORING SYSTEMS

(71) Applicant: Cradlewise Inc., Fremont, CA (US)

(72) Inventors: Pawan Kumar Yadav, Sikar (IN); Bharath Patil, Fremont, CA (US); Gaurav Parashar, Ranchi (IN); Vijayraj Madhav Ghadge, Beed (IN)

(21) Appl. No.: 19/077,082

(22) Filed: Mar. 12, 2025

Related U.S. Application Data
(60) Provisional application No. 63/567,434, filed on Mar. 20, 2024.

(51) Int. Cl. A47D 9/00; A61B 5/00; A61B 5/08

(52) U.S. Cl. CPC A47D 9/00; A61B 5/0077; A61B 5/0816

(57) ABSTRACT

Detecting presence or absence of a baby in an associated cradle. A digital processing system generates data records with each data record having an image and a corresponding label indicating whether baby is present or absent in the cradle corresponding to the image. Each image of a corresponding data record is fed to a first teacher model to cause the first teacher model to infer whether baby is present or absent in the cradle. If the first teacher model infers with a first desired accuracy, the first teacher model is thereafter used as an operative model, or else the student model is used as the operative model. The operative model thus selected is used to infer whether baby is present or absent based on corresponding received images. The first student model is formed by knowledge distillation from the teacher model.

### PRIORITY CLAIM AND RELATED APPLICATION

The instant patent application claims priority from co-pending US provisional patent application entitled, “Automated Local Learning for Accurate and Efficient Baby Detection in Unique Cradle Environments”, Application No. 63/567,434, Filed: 20 Mar. 2024, Attorney docket no.: CRDL-005-USPR, naming Pawan Kumar Yadav, et al as the inventors, and is incorporated in its entirety herewith, to the extent not inconsistent with the content of the instant application.

### BACKGROUND

### Technical Field

Embodiments of the present disclosure relate generally to cradles and more specifically to detecting presence or absence of babies in cradles using monitoring systems.

### Related Art

Cradles are well known in the relevant arts. A cradle generally contains a hammock for holding a baby and can aid in various objectives such as putting the baby to sleep, entertaining the baby when awake, etc. As used herein, a baby refers to children (including toddlers, etc.) of early age who would use cradle for resting.

A cradle may additionally have an associated baby monitoring system directed to monitoring a baby placed in the cradle. Cradles are often designed to provide different actions (or inaction) depending on whether a baby is present or absent in the cradle. For example, only when a baby is present, a monitoring system may cause the cradle to be rocked and/or music may be played in order to soothe the baby back to sleep.

However, challenges are presented in such detection of presence/absence given that other similar weight objects (e.g., toys) may also be placed in the cradle, printed bedsheets with baby-like images may be used, weight type characteristics of baby themselves gradually change, etc.

Aspects of the present disclosure are directed to detecting presence or absence of a babies in cradles.

### BRIEF DESCRIPTION OF THE VIEWS OF DRAWINGS

Example embodiments of the present disclosure will be described with reference to the accompanying drawings briefly described below.

FIG. 1 is a block diagram illustrating an example environment (computing system) in which several aspects of the present disclosure can be implemented.

FIG. 2 is a block diagram illustrating the details of a cradle in association with which several aspects of the present disclosure are implemented (in a baby monitoring system).

FIG. 3 is a flow-chart illustrating the manner in which a baby monitoring system determines whether baby is present or absent in the cradle, in an embodiment of the present disclosure.

FIG. 4 is a block diagram illustrating the implementation details of a baby monitoring system in an embodiment of the present disclosure.

FIG. 5A is a block diagram illustrating the implementation details of a data analyzer in an embodiment of the present disclosure.

FIG. 5B is a timing diagram (not to scale) illustrating the manner in which data records are generated in an embodiment of the present disclosure.

FIG. 5C is a block diagram illustrating the implementation details of a model manager in an embodiment of the present disclosure.

FIG. 6 is a block diagram illustrating the implementation details of a central model controller in an embodiment of the present disclosure.

FIG. 7A is a diagram illustrating the architecture of a teacher model in an embodiment of the present disclosure.

FIG. 7B is a diagram illustrating the architecture of a student model in an embodiment of the present disclosure.

FIG. 8 is a timing diagram illustrating various scenarios for baby detection using the teacher model and the student model, in an embodiment of the present disclosure.

FIG. 9 is a block diagram illustrating the details of digital processing system in which various aspects of the present disclosure are operative by execution of appropriate executable modules.

In the drawings, like reference numbers generally indicate identical, functionally similar, and/or structurally similar elements. The drawing in which an element first appears is indicated by the leftmost digit(s) in the corresponding reference number.

### DETAILED DESCRIPTION

### 1. Overview

Aspects of the present disclosure are directed to a digital processing system for detecting presence or absence of a baby in an associated cradle. The digital processing system generates a first plurality of data records with each data record having an image and a corresponding label indicating whether baby is present or absent in the cradle corresponding to the image. Each image of the first plurality of data records is fed to a first teacher model to cause the first teacher model to infer whether baby is present or absent in the cradle. If the first teacher model infers with a first desired accuracy, the first teacher model is thereafter used as an operative model to infer whether baby is present or absent based on corresponding received images. Otherwise, a first student model is used as the operative model thereafter to infer whether baby is present or not based on corresponding received images. The first student model is formed by knowledge distillation from the teacher model.

According to another aspect, the generation of the first plurality of data records is performed using an upfront teacher model as the operative model when the digital processing system is deployed upfront upon installation of the cradle.

According to one more aspect, the first student model is fine-tuned using a first set of training records prior to using the first student model as the operative model. It may be appreciated that computational complexity is lower for training a student model (compared to teacher model), and accuracy of the student model is enhanced by having the training records correspond to those generated at the associated cradle itself.

According to yet another aspect, the first teacher model and the first student model are received after the generation of the first plurality of data records. Assuming the first teacher model does not infer with the first desired accuracy, the first set of training records correspond to a first subset of the first plurality of data records in performing the fine-tuning of the first student model. In an embodiment, after such fine-tuning, some of the remaining ones of the first plurality of data records are used to check whether the fine-tuned first student model infers with a second desired accuracy. If so, the fine-tuned first student model is thereafter used as the operative model to infer whether baby is present or absent based on corresponding received images.

According to another aspect, the operative model is used for inferring in a first duration following the upfront installation. A second plurality of data records is generated after the first duration, with each data record having an image and a corresponding label indicating whether baby is present or absent in the cradle corresponding to the image. A second teacher model and a second student model are retrieved after the first duration. Using the second plurality of data records, it is checked if the second teacher model infers with the first desired accuracy. If yes, the second teacher model is thereafter used as the operative model. Otherwise, it is checked whether the second student model infers with the second desired accuracy. If not, the second student model is fine-tuned with a third subset of the second plurality of data records until the second student model infers with the second desired accuracy. The fine-tuned second student model is thereafter used as the operative model to infer whether baby is present or absent based on corresponding received images.

According to one more aspect, the first teacher model is fine-tuned at the central server to generate the second teacher model. The first teacher model is shared by a plurality of the digital processing systems prior to the first duration, and the second teacher model is shared by the plurality of digital processing systems after the first duration. Each digital processing system of the plurality of digital processing systems has a respective associated fine-tuned model stored at the central server, wherein the second student model is the corresponding fine-tune model for the digital processing system stored at the central server.

Several aspects of the disclosure are described below with reference to examples for illustration. It should be understood that numerous specific details, relationships, and methods are set forth to provide a full understanding of the disclosure. One skilled in the relevant arts, however, will readily recognize that the disclosure can be practiced without one or more of the specific details, or with other methods, etc. In other instances, well-known structures or operations are not shown in detail to avoid obscuring the features of the disclosure.

### 2. Example System

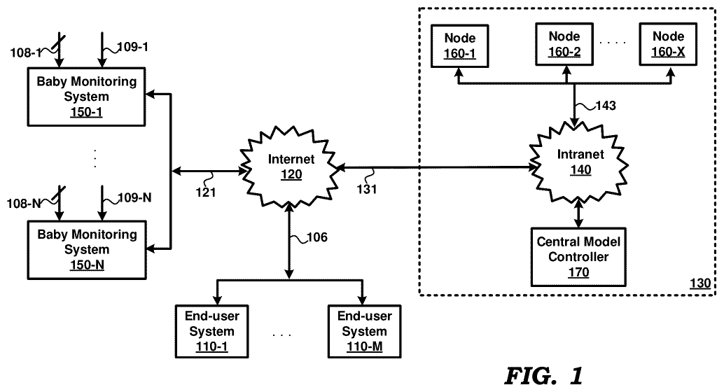

FIG. 1 is a block diagram illustrating an example environment (computing system) in which several aspects of the present disclosure can be implemented. The block diagram is shown containing end-user systems 110-1 to 110-M, baby monitoring systems 150-1 to 150-N and computing infrastructure 130 (M and N representing any natural numbers). Computing infrastructure 130 in turn is shown containing intranet 140, nodes 160-1 through 160-X (X representing any natural number) and central model controller 170. The end-user systems, baby monitoring systems and nodes are collectively or individually referred to by 110, 150 and 160 respectively as will be clear from the context, and similar convention is used for representing other collections of systems also in the present disclosure.

Merely for illustration, only representative number/type of systems are shown in FIG. 1. Many environments often contain many more systems, both in number and type, depending on the purpose for which the environment is designed. Each block of FIG. 1 is described below in further detail.

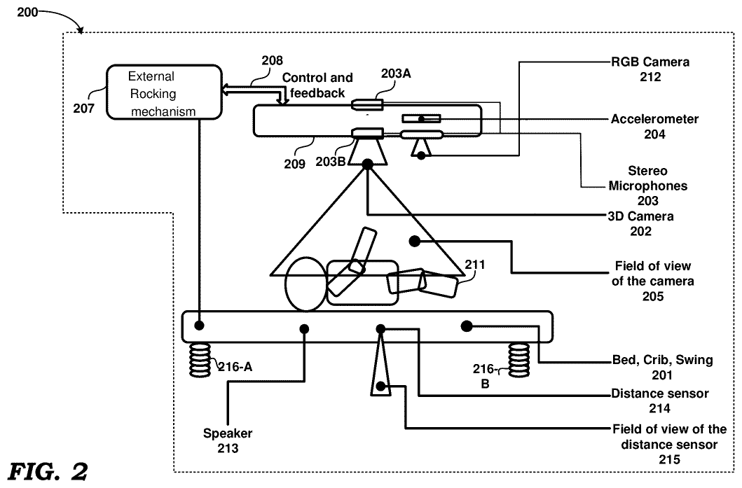

Each of baby monitoring systems (BMS) 150 represents an edge device deployed associated with a corresponding cradle (shown in FIG. 2). BMS 150 operates to monitor the associated cradle via sensors (with the data from sensors received on path 108), detect the presence or absence a baby in the cradle (based on RGB images received on path 109), generate requisite alerts/notifications and trigger actuator(s) as needed.

Each of end-user systems 110 represents a system such as a personal computer, workstation, mobile device/smart phone, computing tablet etc., used by users to monitor babies in cradles by causing the systems to generate (user) requests directed to software applications executing in computing infrastructure 130. A user request refers to a specific technical request (for example, Universal Resource Locator (URL) call) sent to a server system from an external system (here, end-user system) over Internet 120, typically in response to a user interaction at end-user systems 110. The user requests may be generated by users using appropriate user interfaces (e.g., web pages provided by an application executing in a node, a native user interface provided by a portion of an application downloaded from a node, etc.).

In general, an end-user system requests a software application for performing desired tasks and receives the corresponding responses (e.g., web pages) containing the results of performance of the requested tasks. The web pages/responses may then be presented to a user by a client application such as the browser. Each user request is sent in the form of an IP packet directed to the desired system or software application, with the IP packet including data identifying the desired tasks in the payload portion. One or more of end-user systems 110 may be paired with a corresponding BMS 150 to enable communication from BMS 150 to be sent to the user location so that status of a baby can be checked from the user location.

Computing infrastructure 130 is a collection of nodes (160) that may include processing nodes, connectivity infrastructure, data storages, administration systems, etc., which are engineered to together host software applications. Computing infrastructure 130 may be a cloud infrastructure (such as Amazon Web Services (AWS) available from Amazon.com, Inc., Google Cloud Platform (GCP) available from Google LLC, etc.) that provides a virtual computing infrastructure for various customers, with the scale of such computing infrastructure being specified often on demand.

Alternatively, computing infrastructure 130 may correspond to an enterprise system (or a part thereof) on the premises of the customers (and accordingly referred to as “On-prem” infrastructure). Computing infrastructure 130 may also be a “hybrid” infrastructure containing some nodes of a cloud infrastructure and other nodes of an on-prem enterprise system.

All the nodes (160) of computing infrastructure 130 are assumed to be connected via intranet 140. Internet 120 extends the connectivity of these (and other systems of the computing infrastructure) with external systems such as baby monitoring systems 110 and end-user systems 105. Each of intranet 140 and Internet 120 may be implemented using protocols such as Transmission Control Protocol (TCP) and/or Internet Protocol (IP), well known in the relevant arts.

In general, in TCP/IP environments, a TCP/IP packet is used as a basic unit of transport, with the source address being set to the TCP/IP address assigned to the source system from which the packet originates and the destination address set to the TCP/IP address of the target system to which the packet is to be eventually delivered. An IP packet is said to be directed to a target system when the destination IP address of the packet is set to the IP address of the target system, such that the packet is eventually delivered to the target system by Internet 120 and intranet 140. When the packet contains content such as port numbers, which specifies a target application, the packet may be said to be directed to such application as well.

Some of nodes 160 may be implemented as corresponding data stores. Each data store represents a non-volatile (persistent) storage facilitating storage and retrieval of enterprise by software applications executing in the other systems/nodes of computing infrastructure 130. Each data store may be implemented as a corresponding database server using relational database technologies and accordingly provide storage and retrieval of data using structured queries such as SQL (Structured Query Language). Alternatively, each data store may be implemented as a corresponding file server providing storage and retrieval of data in the form of files organized as one or more directories, as is well known in the relevant arts.

Some of the nodes 160 may be implemented as corresponding server systems. Each server system represents a server, such as a web/application server, constituted of appropriate hardware executing software applications capable of performing tasks requested by end-user systems 110 and BMSs 150. A server system receives a request from an end-user system or BMS 150 and performs the tasks requested in the request. A server system may use data stored internally (for example, in a non-volatile storage/hard disk within the server system), external data (e.g., maintained in a data store) and/or data received from external sources (e.g., received from a user) in performing the requested tasks. The server system then sends the result of performance of the tasks to the requesting end-user system/BMS (one of 110/150) as a corresponding response to the request. The results may be accompanied by specific user interfaces (e.g., web pages) for displaying the results to a requesting user.

Central model controller 170 represents a server system which aids in the detection of presence or absence of a baby in the corresponding associated cradle by BMSs 150. As noted above, there is a general need to detect the presence or absence of a baby in a cradle as accurately as possible.

Aspects of the present disclosure are directed to using machine learning (ML) models effectively for such detection (generally referred to as baby detection henceforth). In a prior approach, a shared ML model is used for baby detection across all BMSs 150. However, deploying a shared ML model for universal baby detection across different cradles poses challenges due to the unique characteristics of each cradle, such as toys, bedding, clothing, lighting conditions, camera angles, background clutter, etc. Such characteristics may also change with growth of the baby, thereby negatively impacting the accuracy of a shared ML model. Yet, training extensive deep learning models on edge devices, such as BMS 150, may be difficult and impractical due to limited computational power and memory constraints on the edge devices.

BMS 150 implemented according to aspects of the present disclosure address such challenges. However, it should be appreciated that each BMS 150 may need to be implemented in conjunction with an associated cradle and accordingly the description is continued to illustrate an edge device deployed associated with a cradle, in an embodiment of the present disclosure.

### 3. Edge Device in Association with a Cradle

FIG. 2 is a diagram illustrating the details of a cradle, associated with baby monitoring system is implemented according to aspects of the present disclosure. Cradle 200 is shown containing a bed/crib/swing 201, fixed frame 209 and external rocking mechanism 207. Crib 201, in which baby 211 may be placed, may be suspended from fixed frame 209 (the mechanism for such suspension not shown in FIG. 2), and may be designed to be able to rock or oscillate about one or more axes. External rocking mechanism 207 is used to rock/swing crib 201 in desired modes of operation (vertical rocking and/or horizontal rocking). An example implementation of external rocking mechanism 207 is described in the patent U.S. Pat. No. 10,357,117B2, which is incorporated its entirety herewith.

Fixed frame 209 may be connected to crib 201 or ceiling or floor of a room, or any other stable surface by suitable means, not shown, and anchors the cradle to a stable surface. In an embodiment, when cradle 200 is rocked, frame 209 and crib 201 move together as a single unit such that the field of view of 3D camera remains unaltered (relative to crib 201) with the rocking movements. Fixed frame 209 and crib 201 may house the components/sensors that collect/capture data in/around cradle 200. However, in alternative embodiments, the components/sensors/sub-systems of cradle 200 may be placed suitably elsewhere in the cradle. Though the connection paths are not shown in FIG. 2, all of the signals from various sensors are received by BMS 150 on path 108, and BMS 150 drives the requisite components (e.g., RGB camera) and actuators, as described in sections below.

Fixed frame 209 houses accelerometer 204, an RGB camera 212, a 3D (three dimensional) camera 202 and stereo microphones 203. Crib 201 houses speaker 213, distance sensor 214 and springs 216. Although not shown, cradle 200 also includes power sources (e.g., battery) and electronics/communications/processing blocks/alert generation units (as well as non-volatile memory for storing the software instructions implementing the various blocks described herein) for performing various operations described herein. Additionally, cradle 200 may contain a music system. It may thus be appreciated that 3D camera 202, processing block(s) and alert generator are part of a unit (here fixed frame 209) placed in the vicinity of the baby (here baby 211 is within the field of view of 3D camera, which is a part of fixed frame 209 itself). Thus, the components are all placed to be within a short distance (say utmost 10 meters, but typically more in the range of 1-2 meters), thereby providing a solution for the detection system individually (instead of a centralized or remote solution for monitoring many babies at different locations).

Stereo microphones 203 include at least a pair of microphones 203A and 203B. As is well known in the relevant arts, difference in time of arrival of sound at microphones 203A and 203B represents the angle of arrival of sound. As is also well known in the relevant arts, stereo microphones cannot discern between sounds originating from any point on a given cone whose axis passes through line joining the two microphones. The size of the cones gets smaller in the direction of the axis and gets biggest in the plane perpendicular to the axis. Hence it is important to have the subject near the line that joins the two microphones. Accordingly, the baby (and therefore crib 211) is placed in a location that is in the line joining the microphones 203A and 203B, as can also be observed from FIG. 2. Such orientation of the microphones enables a better rejection of all other sounds in the environment. Stereo microphones 203 generate electrical (e.g., digital) representation of the sound/noises in/around cradle 200.

Speaker 213 generates sound signals based on a received control input (e.g., an electrical/digital value). The control input may be received from, for example, a music system (fitted in cradle 200) for entertainment of babies.

Accelerometer 204 is designed to capture motion information inside cradle 200. As is well known in the relevant arts, an accelerometer takes in as input a mechanical movement (force applied to a mass included in the sensor) and generates as output analog voltage signal(s) that varies directly with the applied mechanical movement. In an embodiment, accelerometer 204 is implemented as one or more MEMS (micro electromechanical system) sensor(s) that is/are capable of sensing the motion inside cradle 200. Accelerometer 204 generates corresponding analog voltage output corresponding to the acceleration measured along the X, Y, and Z axes.

RGB camera 212 is designed to capture 2D images with color information of objects/scene in its field of view (not shown in FIG. 2), and is implemented in an embodiment to be sensitive to visible light. However, in other embodiments an RGB camera sensitive to other spectral regions of light can be used instead. Each RGB image contains multiple pixels/pixel locations, with each pixel specifying the red, green, and blue color values of the point/area represented by the pixel. A raw RGB image is an uncompressed image that consists of RGB values obtained at each pixel.

3D camera 202 is designed to capture 3D images of objects/scene in its field of view (FoV) 205, and is implemented in an embodiment to be sensitive to light in the near-infrared spectrum. However, in other embodiments a 3D camera sensitive to other spectral regions of light can be used instead. Each 3D image contains multiple pixels/pixel locations, with each pixel specifying the intensity (color, black/white, etc.) of the point/area represented by the pixel. As noted above, each pixel is also characterized by coordinates specifying spatial location of the corresponding point/area of the object (infant) represented by the pixel. 3D camera provides a point cloud that consists of (X, Y, Z) data for each pixel and a 2D (2-dimensional) monochrome image which consists of the intensity data for each pixel. A raw 2D image is an image that consists of the intensity obtained at each pixel in the 3D camera and looks similar to an image obtained from a regular monochrome sensor/camera with a flash light or a black and white version of a color photo obtained using a regular camera and flash light. 3D camera 202 generates one or more frames (i.e., a sequence of successive frames of) of raw 3D images of a scene including baby 211 in FoV 205. It is noted here that, depending on the nature of the next stage(s) of processing, the term ‘raw 3D image’ can refer to either a single frame of pixels representing a scene/object in FoV 205, or a sequence of successive images of frames at corresponding instants of time.

As is well known in the relevant arts, 3D images provide depth perception, in addition to height and width. In an embodiment, 3D camera 202 uses a Time-of-Flight (ToF) sensor, well known in the relevant arts. A ToF sensor illuminates the field of view 205 of the 3D camera with one or more pulses of light (e.g., visible, near-infrared etc.). Reflection from objects in the field of view 205 that are at different distances (depths) from the ToF sensor reach the ToF sensor at different times, which is then used to determine the distances (depth component for each pixel) to the corresponding objects or portions of the scene (here infant 211 and possibly a portion of crib 201). In addition, 2D image of the scene is also obtained, disregarding the depth component.

In an alternative embodiment, 3D camera 202 is implemented using two image sensors positioned side-by-side for capturing simultaneous images of the scene/object from slightly different angles, as is also well known in the relevant arts. In each embodiment, 3D camera 202 may also include illumination sources and optical systems.

Distance sensor 214 is designed to capture distance from the sensor to an object/article of interest. In an embodiment, distance sensor 214 is placed in the bottom of crib 201 facing towards the surface on which cradle 200 is kept (as depicted in FIG. 2), and captures the distance from the sensor to the surface (e.g., floor), and is implemented as an ultrasonic sensor in an embodiment. As is well known in the relevant arts, upon receipt of a trigger (e.g., due to placement of an object/baby inside cradle 200), ultrasonic sensor generates and emits ultrasonic pulses that are reflected back towards the sensor by an object (here floor) that is within field of view 215 of the sensor. The output of distance sensor 214 is a digital pulse with a width directly proportional to the measured distance. Although the illustrative embodiment describes distance sensor 214 as being implemented as an ultrasonic sensor, distance sensor 214 in alternative embodiments may implemented differently. The output of distance sensor 214 is used to estimate the mass of cradle 200, as will be described below in detail.

The manner in which baby monitoring system detects the presence or absence of a baby in a cradle is described below with examples. The examples refer to ‘teacher model’ and ‘student model’, which are both described below first.

### 4. Teacher Model and Student Model

According to an aspect of the present disclosure, central model controller 170 forms a compact baby detection model suitable for edge devices such as BMS 150. In an embodiment, central model controller 170 forms a teacher model by training the model based on data records collected globally from various cradles, and forms a base student model from the trained teacher model using knowledge distillation techniques. The base student model is thus ‘pre-trained’ with global data records. In an embodiment, central model controller 170 forms the teacher model and the student model using convolutional neural network (CNN) architecture.

As is well known in the relevant arts, knowledge distillation (KD) is a machine learning technique that transfers knowledge (i.e., the weights and biases used in CNN) from a larger model (here, the teacher model) to a smaller model (here, the student model). In an embodiment, central model controller 170 employs feature-based KD in which the internal representations (e.g., feature embeddings) of the teacher model are extracted from the intermediate layers of CNN, and the student model is trained to mimic the features learned by the teacher model. The manner in which the teacher model and the student model are formed is described in further detail in sections below.

The base student model is fine-tuned at each BMS 150 using data collected locally at that particular associated cradle. Central model controller 170 maintains a repository of student models for corresponding BMSs 150.

The manner in which baby monitoring system detects the presence or absence of a baby in a cradle is described next.

### 5. Flow-Chart

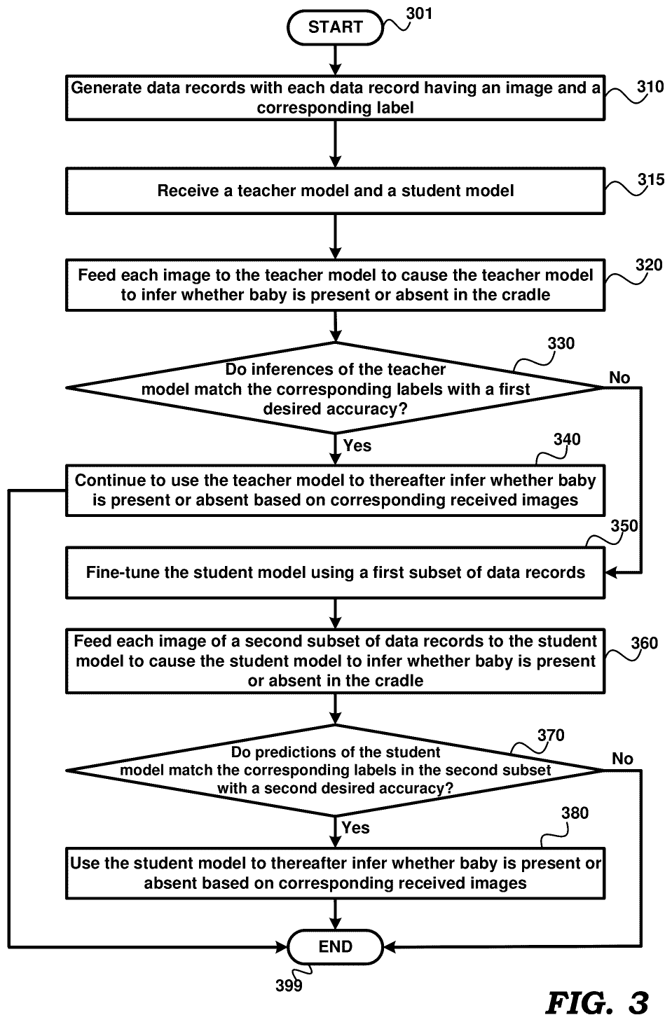

FIG. 3 is a flow-chart illustrating the manner in which detection of presence or absence of baby in a cradle is performed by baby monitoring system (BMS) 150 according to aspects of the present disclosure. The flow-chart is described with respect to the systems of FIGS. 1 and 2, in particular BMS 150, merely for illustration. However, many of the features can be implemented in other environments also without departing from the scope and spirit of several aspects of the present invention, as will be apparent to one skilled in the relevant arts by reading the disclosure provided herein.

In addition, some of the steps may be performed in a different sequence than that depicted below, as suited to the specific environment, as will be apparent to one skilled in the relevant arts. Many of such implementations are contemplated to be covered by several aspects of the present invention. The flow chart begins in step 301, in which control immediately passes to step 310.

In step 310, BMS 150 generates data records with each data record having an image and a corresponding label indicating whether baby is present or absent in the cradle corresponding to the image. BMS 150 generates such data records based on the captured images and data collected by sensors (such as accelerometer 204, 3D camera 202, distance sensor 214, etc.) associated with the cradle. Thus, each label instance is generated for sensor data received at a time instance, and the generated label instance is associated with an image captured at (substantially) same time instance to form a corresponding data record.

In step 315, BMS 150 receives a teacher model and a student model from central model controller 170.

In step 320, BMS 150 feeds each image of each data record to the teacher model to cause the teacher model to infer whether baby is present or absent in the cradle. Thus, the teacher model takes an ‘image’ (of a data record) as an input and provides as an output a corresponding label—‘baby present’ or ‘baby absent’, classifying the input image.

In step 330, BMS 150 checks whether each inference of the teacher model matches the corresponding label of the same record as a basis to determine whether the teacher model infers with a first desired accuracy. Accuracy refers to the measure of correct inferences made by the model, and may be calculated as the number of correct inferences divided by total/all inferences for a given test dataset. The accuracy of an ML model is expressed as a percentage as is well known in the relevant arts. Thus, if a model correctly infers labels for 9 out of every 10 inputs, then the model is said to be 90% accurate. If the first desired accuracy is satisfied by the teacher model (value “YES”), control passes to step 340, and to step 350 otherwise.

In step 340, BMS 150 continues to use the teacher model to thereafter infer whether baby is present or absent based on corresponding images received during normal operations.

In step 350, BMS 150 fine-tunes a student model using a first subset of the data records noted above. The term ‘fine-tunes’ refers to the process of modifying/updating the pre-trained (at central model controller 170) student model using the first subset as new data. The first subset of data records may thus be viewed as ‘training data’. BMS 150 fine-tunes the student model based on training data collected from the local environment (the associated cradle), allowing the student model to adapt to conditions and variations specific to the cradle associated with BMS 150. Adaptation of the student model to handle diverse cradle-specific characteristics may improve the accuracy and reliability of baby detection.

In step 360, BMS 150 feeds each image of a second subset of data records to the fine-tuned student model to cause the fine-tuned student model to infer whether baby is present or absent in the cradle. Thus, the student model, similar to the teacher model, takes an ‘image’ as an input and provides as an output a corresponding label—‘baby present’ or ‘baby absent’, classifying the input image.

In step 370, BMS 150 checks whether inferences of the student model match the corresponding labels with a second desired accuracy, similar to the techniques described above with respect to step 330. If the second desired accuracy is satisfied by the student model (value “YES”), control passes to step 380, and to step 399 otherwise.

In step 380, BMS 150 uses the fine-tuned student model to thereafter infer whether baby is present or absent based on corresponding images received during normal operations. Though not shown in the flow-chart, BMS 150 sends model information of the fine-tuned student model to central model controller 170. The same fine-tuned model may be received later when requested by BMS 150. Control passes to step 399, where the flow-chart ends.

It is noted herein that the flow-chart of FIG. 3 is operative in a first duration following upfront installation of BMS 110 at the cradle. Upfront installation generally refers to the instance when a BMS is deployed for the first time associated with a new cradle.

It may be appreciated that fine-tuning a pre-trained student model is computationally less expensive than fine-tuning a teacher model. Accordingly, the student model is fine-tuned on the edge device having limited storage and computational power. In addition, baby detection accuracy may be improved since the student model is fine-tuned using data local to the cradle.

Thus, the flow-chart of FIG. 3 operates to detect the presence or absence of a baby in a cradle. While the (operative) model thus used may infer presence or absence of a baby in a cradle with a desired level of accuracy, various changes noted above may cause the operative model to fail that criteria over time. Accordingly, it may be necessary to periodically update the operative model.

### 6. Periodic Update of Operative Model

While the operative model (either teacher model per step 340 or student model per step 380) continues to infer presence or absence of a baby in cradle 200, additional data records are generated concurrently periodically (e.g., every 10 minutes each). Thus, the images captured and the label formed based on status determined by the sensors, are compiled as additional data records. The previous records of the first duration may be discarded/deleted and these additional data records alone form the basis for further processing as described below.

Specifically, BMS 150 may perform steps 310 onwards using such additional data records. Specifically, the models may be retrieved again (from central model controller 170). For convenience, the retrieved models are referred to as TM-2 and SM-2. Teacher model TM-2 is checked for accuracy (using the additional data records) in steps 320 and 330. Teacher model TM-2 is used as the operative model if the desired accuracy is satisfied in step 330.

However, if the first desired accuracy is not satisfied by the teacher model (value “NO”) in step 330, BMS 150 checks if the student model (retrieved from central model controller 170, SM-2) meets the desired second accuracy. Such check is performed by BMS using a first subset of data records generated in the first duration. It may be observed that SM-2 may correspond to the fine-tuned student model stored in central model controller 170 (as noted above). Only if the second desired accuracy is not satisfied by student model SM-2, BMS 150 fine-tunes student model SM-2 using a subset of the data records generated in the first duration until the fine-tuned student model (referred to as FT-SM-2 for convenience) infers with the second desired accuracy.

In other words, in comparison to the flow-chart of FIG. 3, accuracy is checked first and then fine-tuning is performed only if required given that the student model is already fine-tuned in the first duration.

As noted above, while the fine-tuned model (SM-2) was previously satisfying the desired accuracy, developmental changes in the baby (e.g., baby started sleeping on sides instead of on back) and/or changes in cradle (e.g., use of printed bedsheets with baby-like images, etc.) may cause SM-2 to not satisfy the desired accuracy. If the recently fine-tuned student model (FT-SM-2) satisfies the desired accuracy, the operative model is replaced with the fine-tuned student model (FT-SM-2).

Thus, periodic update of the operative model may enable baby detection as accurately as possible.

The description is continued with respect to an example implementation of a baby monitoring system in an embodiment of the present disclosure.

### 7. Baby Monitoring System

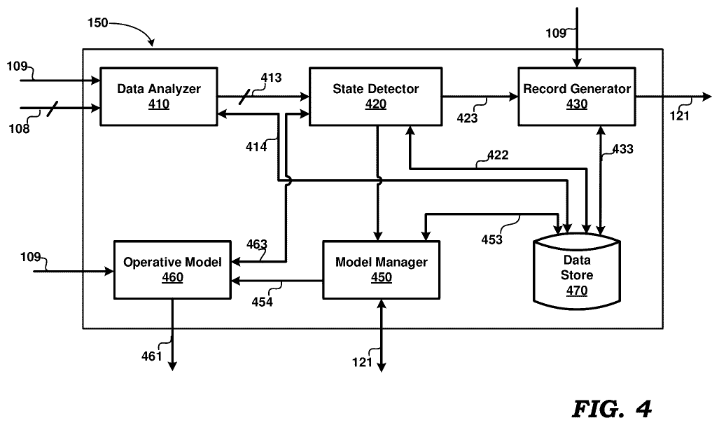

FIG. 4 is a block diagram illustrating the implementation details of a baby monitoring system (150) in one embodiment. The block diagram is shown containing data analyzer 410, state detector 420, record generator 430, model manager 450, operative model 460 and data store 470. Each of the blocks is described in detail below.

Data analyzer 410 receives sensor data on path 108 and raw RGB images on path 109. In an embodiment, sensor data received on path 108 includes raw 3D images and depth data received from 3D camera 202, analog voltage signal (representing motion data) received from accelerometer 204, and digital pulses (representing distance data) received from distance sensor 214. Data analyzer 410 pre-processes the raw data received from various sensors, filters out any irrelevant information (such as noise, etc.) in a known way, and generates measurements such as mass of cradle, monitored respiratory rate, vertical movement of cradle present or not, and activity in/around cradle present or not, on path 413.

Data analyzer 410 stores the received RGB images in data store 470. Data analyzer 410 sends a trigger to RGB camera 212 (via corresponding means not shown in FIG. 4) to capture an RGB image, and stores the RGB image (along with the timestamp of receipt of the image) received in response to the trigger in data store 470. In an embodiment, data analyzer 410 triggers capture of an RGB image at preset time points (e.g., once in every 10 minutes) or when movement is detected within cradle 200 (as indicated by sensor data received from 3D camera 202 and accelerometer 204).

Operative model 460 represents an ML model that infers whether baby is present or absent in the cradle based on images received on path 109 during normal operations, with the output being provided on path 461. In an embodiment, operative model receives an RGB image as input and generates a label (‘baby present’ or ‘baby absent’) for the received RGB image along with a confidence score. During data record generation process, operative model 460 receives RGB images from state detector 420 and generates the label and confidence score corresponding to the images (via path 463).

State detector 420 receives measurements from data analyzer 410 on path 413, inference from operative model 460, and generates state indicators indicating ‘baby present’ and ‘baby absent’ states. In an embodiment, the state detector 420 generates state indicators as output tuples, with each tuple containing two timestamps: the start time (ST) and the end time (ET), along with the corresponding state-‘baby present’ (BP) or ‘baby absent’ (BA). Thus, a tuple [BP-ST, BP-ET, ‘baby present’] indicates that a baby was present in cradle 200 from the start time (BP-ST) to the end time (BP-ET), while a tuple [BA-ST, BA-ET, ‘baby absent’] indicates that baby was not present in cradle 200 from the start time (BA-ST) to the end time (BA-ET). State detector 420 stores the generated tuples in data store 470 (via path 422). The manner in which state detector 420 determines ‘baby present’ and ‘baby absent’ states in an embodiment is described in the sections below.

Record generator 430 receives (via path 423) state indicators from state detector 420, raw RGB images from camera 212 on path 109, and generates data records on path 433 to be stored in data store 470. Record generator 430 labels each received RGB image as ‘baby present’ or ‘baby absent’ based on ‘baby present’ and ‘baby absent’ state indicators received from block 420. Thus, each data record generated by record generator 430 contains an image and a corresponding label (indicating whether baby is present or absent in cradle 200) corresponding to the image. Details about record generation are described in further detail in sections below.

Data store 470 represents a non-volatile (persistent) storage facilitating storage and retrieval of data used by other components of BMS 150 (such as data analyzer 410, record generator 430, etc.). Data store 470 may be implemented as a database server using relational database technologies and accordingly provide storage and retrieval of data using structured queries such as SQL (Structured Query Language). Alternatively or in addition, data store 470 may be implemented as a file server providing storage and retrieval of data in the form of files organized as one or more directories, as is well-known in the relevant arts.

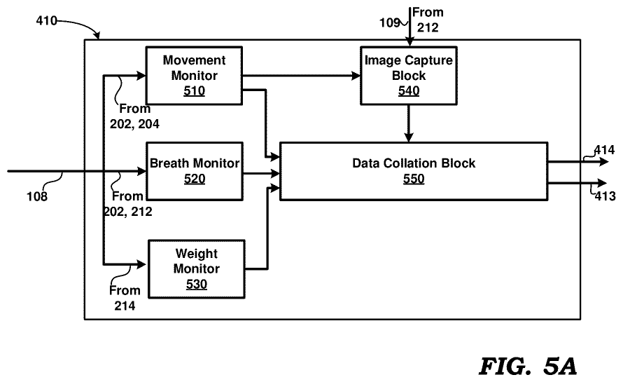

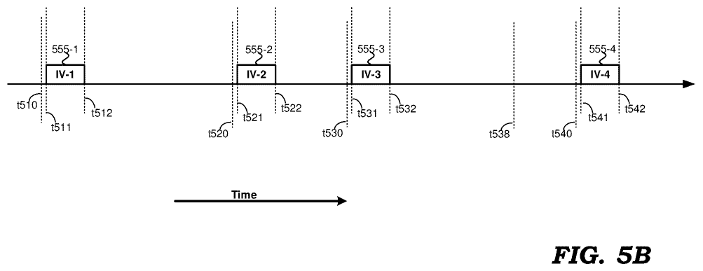

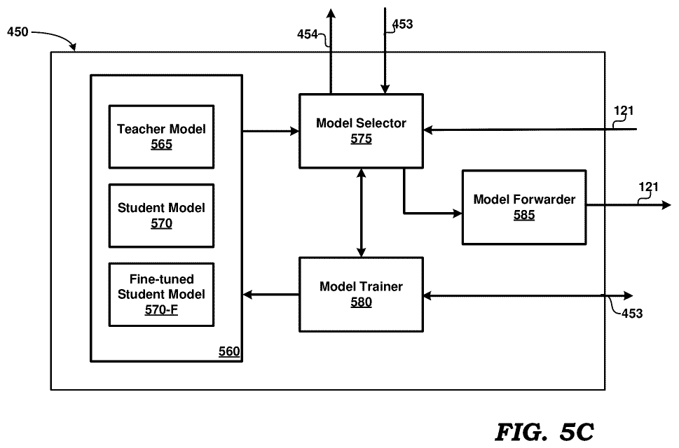

Model manager 450 receives the teacher model and student model from central model controller 170 on path 121, and operates to update operative model 460 (via path 454) as described above. Specifically, model manager 450 evaluates the accuracy of the received models, fine-tunes the student model if required, and updates operative model 460 based on the result of the evaluation(s). An example implementation of model manager 450 is described in detail with respect to FIG. 5C.

The description is continued to illustrate the implementation details of a data analyzer in an embodiment of the present disclosure.

### 8. Data Analyzer

FIG. 5A is a block diagram illustrating an example implementation of a data analyzer (410) in an embodiment of the present disclosure. The block diagram is shown containing movement monitor 510, breath monitor 520, weight monitor 530, image capture block 540 and data collation block 550. Each of the blocks is described in detail below.

Movement monitor 510 receives raw distance data of the object/scene in FoV 205 from 3D camera 202 and analog voltage outputs from accelerometer 204. Movement monitor 510 processes the raw distance image(s) using well known techniques to obtain a ‘depth image’ (3D image) consisting of x, y and z coordinates of each point in the scene in field of view 205 as well as the intensity (I) of the corresponding point. The 3D image is then used to determine horizontal and vertical movement in/around cradle 200. Movement monitor 510 also categorizes the movement as originating from within cradle 200 or from outside cradle 200 based on data received from ToF sensor.

Specifically, a 3D image of an empty cradle (i.e., without the baby or any other object inside the cradle) is saved in data store 470 upon upfront installation of the cradle. The 3D image consists of a boundary of cradle 200 and an inner area. The dimensions of boundary and the inner area are known apriori. The depth information of 3D images generated based on received data from camera 202 is compared with that of 3D image of the empty cradle. Variations in depth in the inner area of cradle are considered as “inner movements” while variations in depth at the boundary of cradle are considered as “boundary movements”.

Movement monitor 510 filters certain specific frequencies in output of accelerometer 204 to remove noise and to minimize the impact of small, insignificant movements (e.g., to reduce the effects of cradle (or crib) motion on baby motion.). By calculating the energy of the resulting time signal, information about the movement is extracted. If the movements are greater than preset thresholds (received from data store 470), movement monitor 510 determines that movement is present inside cradle 200. If movement is present, movement monitor 510 continues to analyze ToF sensor data for a pre-determined time interval (e.g., 3 seconds). If movement is determined to be present throughout the pre-determined time interval and such movement is determined to be ‘boundary movement’ noted above, movement monitor 510 ascertains the occurrence of “an intervention” at the cradle. An intervention as used herein refers to an external object interacting with the cradle (e.g., parents placing the baby (hereafter baby placement activity) or retrieving the baby (hereafter baby retrieval activity) or maintenance activity such as cleaning/arranging blankets, etc.). The time instance of start of boundary movement is referred as “intervention start-time”, and the time instance of end of boundary movement (based on ToF sensor data) is referred to as “intervention end-time”. Thus, each intervention duration is bounded by respective start-time and end-times. A typical intervention duration is around 20 seconds to 1 minute. Movement monitor 510 outputs intervention start-time and intervention end-time for each determined intervention.

Breath monitor 520 receives raw 3D images from 3D camera 202 and RGB video from RGB camera 212. Each frame in RGB video is divided into multiple regions to enable localized motion analysis, allowing for a focused examination of specific areas likely associated with breathing-related movements (e.g., chest, torso and neck in this case). To optimize computational efficiency and enhance accuracy, ToF depth data 202 is used to exclude regions without objects or significant depth variations, ensuring processing is focused on areas where potential breathing signals may exist.

In an embodiment, breath monitor 520 applies Principal Component Analysis (PCA) to the optical flow data within the remaining regions, identifying dominant motion patterns while effectively filtering out noise and irrelevant movements. To further refine the analysis, a bandpass filter with a frequency range corresponding to typical human respiratory rates (e.g., 5 to 50 breaths per minute) is applied to the PCA results for each region. Such filtering eliminates high-frequency noise and low-frequency disturbances unrelated to breathing. The PCA results from all selected regions are subsequently combined into a unified dataset. A secondary application of PCA is performed on the consolidated dataset to further isolate the most significant motion patterns indicative of breathing across the entire frame. Finally, periodic peaks corresponding to breathing motions are detected from the consolidated PCA data, and the respiratory rate (RR) in breaths per minute is calculated based on the detected peaks.

Weight monitor 530 receives digital pulses from distance sensor 214 representing distance of crib 201 from the floor, and generates a value indicating mass (in kilograms, kg) of cradle 200. The mass of an empty cradle (i.e., including various sensors/actuators, etc. but excluding external objects/baby placed in cradle 200) is measured as part of an initial calibration process, and the value is stored in data store 470. An increase in mass of cradle 200 (e.g., due to placement of baby in the cradle) results in (i) compression of springs 216 by a certain length, and (ii) a corresponding reduction in distance of sensor 214 from the floor. The weight corresponding to a certain change in length of spring may be calculated in a known manner (e.g., using the spring constant of springs 216 known apriori, and stored in data store 470). Thus, a unit change in distance from the floor corresponds to a certain change in weight of cradle 200. Accordingly, the approximate extra mass present inside cradle 200 is determined by subtracting the mass of the empty cradle from the current calculated mass.

Image capture block 540 operates to capture images from RGB camera 212. In an embodiment, image capture block 540 sends a trigger to RGB camera 212 (via corresponding means not shown in FIG. 5A) to capture an RGB image, and receives (on path 109) the image captured in response to the trigger. In the embodiment, image capture block 540 triggers capture of an RGB image at preset time points (e.g., once in every 10 minutes) or when movement is detected within cradle 200 (as indicated by movement data received from movement monitor 510). When movement is detected within cradle 200, image capture block 540 may trigger capture of images at a higher frequency (e.g., every 2 minutes) as long as the movement persists. Image capture block 540 also triggers capture of images at intervention start-time and intervention end-time (received from block 510).

Data collation block 550 operates to correlate data received from each of blocks 510, 520 and 530 with RGB image(s) received from block 540 in a same time frame/interval. Specifically, when an intervention indication is received from movement monitor 510, data collation block 550 operates to compare sensor data (e.g., mass of cradle, depth, vertical movement, etc.) received just prior to intervention start-time with that received at intervention end-time. Data collation block 550 calculates, in a known way, depth variation by subtracting coordinates and intensity values in depth image acquired just prior to intervention start-time from those in the image obtained at intervention end-time in the area of interest. Data collation block 550 determines, in a known way, whether vertical movement is present in the intervention duration. Data collation block 550 determines mass variation by subtracting mass information acquired just prior to intervention start-time from that obtained at intervention end-time. Data collation block 550 retrieves inner movement data (generated by block 510) and respiratory rate (generated by block 520) for the duration of the intervention from data store 470 (via path 414). Data collation block 550 generates the collated data as corresponding data points on path 413.

An example data point corresponding to an intervention contains values for the following parameters: [Intervention start-time, Intervention end-time, Mass-difference, Depth-difference, Vertical-movement-present (Y/N), Inner-movement, Respiratory-rate, RGB-image-start-time, RGB-image-end-time].

Even in the absence of intervention indication from block 510, data collation block generates data points periodically (e.g., every 5 minutes). An example periodic data point (i.e., not corresponding to an intervention) contains values for the following parameters: [Measurement start-time, Measurement end-time, Inner-movement, Respiratory-rate].

Examination of such correlated data points aids state detector 420 to determine boundaries of time windows in which baby is present or absent in the cradle, as will be described next.

### 9. State Detection

As noted above, state detector 420 generates state indicators as output tuples with each tuple containing two timestamps and the corresponding state-‘baby present’ or ‘baby absent’. In an embodiment, state detector 420 determines whether an intervention corresponds to baby placement activity or baby retrieval activity. If an intervention is determined to correspond to baby placement activity, state detector 420 records the intervention start-time as the end time of ‘baby absent’ state and the intervention end-time as the start time of ‘baby present’ state (since baby was not present in cradle prior to the start of the intervention and is present after the intervention).

On the other hand, if an intervention is determined to correspond to baby retrieval activity, state detector 420 records the intervention start-time as the end time of ‘baby present’ state and the intervention end-time as the start time of ‘baby absent’ state (since baby was present in cradle prior to the start of the intervention and is absent after the intervention).

The manner in which generation of such tuples is performed in an embodiment of the present disclosure is described below.

The terminology/acronyms used in the description are listed below for convenience:
INV: Intervention
DP: Data Point
I-ST: Intervention start-time in DP
I-ET: Intervention end-time in DP
MD: Mass-difference value in DP
DD: Depth difference value in DP
IM: Inner-movement value in DP
RR: Respiratory-rate value in DP
RGB-ST: RGB image captured at intervention start-time
RGB-ET: RGB image captured at intervention end-time
BP-ST: Start time instance of ‘baby present’ state
BP-ET: End time instance of ‘baby present’ state
BA-ST: Start time instance of ‘baby absent’ state
BA-ET: End time instance of ‘baby absent’ state

### Baby Placement Activity

State detector 420 receives a data point (DP1) from data analyzer 410 corresponding to an intervention (INV-1) with the values [I-ST-1, I-ET-1, MD-1, DD-1, Y, IM-1, RR-1, RGB-ST-1, RGB-ET-1]. State detector 420 examines the values in DP1 and the inference received from operative model 460 in order to identify ‘baby placement’ activity. Specifically, state detector 420 first checks whether all of the following conditions are satisfied by values in the data point (i.e., DP1):
A. Mass-difference (MD-1) is a positive value and exceeds a pre-determined threshold
B. Depth-difference (DD-1) is a negative value (indicating a decrease in depth)
C. Vertical-movement-present is ‘Y’

If all conditions noted above are satisfied, state detector 420 sends RGB-ET-1 contained in DP1 to operative model 460.

If operative model 460 infers ‘baby present’ for RGB-ET-1, state detector 420 checks whether inner movement in cradle 200 (based on IM-1 value in DP1) exceeds a pre-determined lower threshold and breathing rate (based on RR-1 value in DP1) exceeds a pre-determined lower threshold, and if so, concludes that the intervention corresponds to a baby placement activity.

When state detector 420 determines baby placement activity, state detector 420 records the time instance of intervention end-time (I-ET-1) of DP1 as start time of ‘baby present’ state (BP-ST) if a previously recorded BP-ST value does not exist. Additionally, state detector 420 records intervention start-time (I-ST-1) as the end time of ‘baby absent’ state (BA-ET), but only if BA-ST value exists. State detector 420 generates the tuple [BA-ST, I-ST-1, ‘baby absent’].

It may be appreciated that in most cases, when parents place the baby in the cradle, they do so gently, ensuring that the body of the baby is in FoV (205) of camera 202, and not entirely covered by clothing. In these instances, operative model 460 is generally successful in detecting the presence of baby in the cradle.

However, if operative model 460 infers ‘baby absent’ for RGB-ET-1 (i.e., inference of operative model 460 does not concur with collated sensor data in DP1), state detector 420 continues to examine data points received from block 410 for a pre-determined time interval (e.g., 4 hours from time instance I-ET-1, ending at time instance T2). In general, the pre-determined time interval may be configured such that the duration is sufficient for state detector 420 to conclusively determine the presence or absence of the baby in cradle.

Based on the data points, state detector 420 performs checks as follows:
1. If neither normal breathing rate (as specified by corresponding high and low thresholds) nor inner movement is detected in duration (I-ET-1 to T2), state detector 420 concludes that baby is not present in the cradle as it is unlikely that the baby remains in the cradle without any detectable motion and/or breathing. Accordingly, state detector 420 records I-ET-1 as the start time of the ‘baby absent’ state (BA-ST), but only if a previously recorded BA-ST value does not exist. Additionally, state detector 420 does not record BP-ST value. State detector 420 does not generate any tuple corresponding to INV-1.
2. If a normal breathing rate is detected in all the data points corresponding to the time interval (I-ET-1 to T2), state detector 420 concludes that INV-1 corresponds to a baby placement activity. State detector 420 accordingly generates the tuple [BA-ST, I-ST-1, ‘baby absent’] as noted above. It may be appreciated that in most case, when baby is present in the cradle and not moving, a normal breathing rate may be detected.
3. If a normal breathing rate is not detected but inner movements are detected in data points corresponding to time interval (I-ET-1 to T2), state detector 420 re-evaluates inferences received from operative model 460, as follows:
   i). If operative model 460 infers ‘baby present’ in more than a threshold percentage (e.g., 80%) of the RGB images captured in the time interval (I-ET-1 to T2), state detector 420 concludes that that INV-1 corresponds to a baby placement activity. State detector 420 accordingly generates the tuple [BA-ST, I-ST-1, ‘baby absent’].
   ii). If operative model 460 infers ‘baby absent’ more than the threshold percentage of the RGB images captured in the time interval (I-ET-1 to T2), state detector 420 concludes that INV-1 does not correspond to baby placement activity. Accordingly, state detector 420 records time instance I-ET-1 as BA-ST if BA-ST value does not exist. Also, state detector 420 records time instance I-ST-1 as BP-ET, but only if BP-ST value exists. State detector 420 generates tuple [BP-ST, I-ST-1, ‘baby present’].

It may be appreciated that in most cases, if a baby is moving in the cradle, operative model 460 accurately infers ‘baby present’. However, if multiple inferences by operative model 460 still indicate ‘baby absent’, it may suggest that the detected movement inside the cradle is caused by something else (such as, for example, a hanging cloth swaying due to a fan or a toy placed inside the cradle that is moving). The above noted technique of determining ‘baby placement’ activity may ensure differentiation between genuine baby movements and other disturbances mimicking baby movements.

iii). If neither of the above conditions (i and ii) is satisfied, state detector 420 considers the case as a ‘confusion’, where it cannot be confidently determined whether the baby is present or not in the cradle. In such a scenario, state detector 420 checks for previously recorded BP-ST and baby BA-ST values. If BP-ST exists, state detector 420 generates tuple [BP-ST, I-ST-1, ‘baby present’]. If BA-ST exists, state detector 420 generates tuple [BA-ST, I-ST-1, ‘baby absent’]. State detector 420 does not record ‘baby present’ or ‘baby absent’ states in the duration from T2 to the immediately subsequent ‘baby placement’ or ‘baby retrieval’.

In an embodiment, when inner movement is detected in cradle 200, state detector 420 applies a blur feature to RGB images given to operative model 460 for inference. Such a technique may ensure that inference is only performed on regions where movement is detected, as the rest of the image is blurred. As a result, operative model 460 focuses on identifying whether the moving object is a specific entity, such as a baby, thereby enabling the operative model to concentrate on the moving object rather than all the items within the cradle. In the embodiment, blur is applied by projecting image pixels (from 3D camera 202) corresponding to inner movement onto the frame captured by RGB camera 212 with both image and frame captured in a substantially same time frame/interval. Convolution is applied to blur the pixels where there is no movement.

It may be appreciated that if state detector 420 has determined a current intervention as corresponding to a ‘baby placement’ activity (i.e., state detector 420 has recorded BP-ST), state detector 420 does not record an immediate subsequent intervention as baby placement activity even though collated sensor data and/or operative model output suggest baby placement. Such a scenario may occur when a baby is actually placed in the cradle as part of a first intervention while a heavy object (e.g., toy or blanket) mimics placement of a baby in an immediately subsequent intervention.

### Baby Retrieval Activity

State detector 420 receives a data point (DP2) from data analyzer 410 corresponding to an intervention (INV-2) with the values [I-ST-2, I-ET-2, MD-2, DD-2, Y, IM-2, RR-2, RGB-ST-2, RGB-ET-2]. State detector 420 examines the values in DP2 and the inference received from operative model 460 in order to identify ‘baby retrieval’ activity. Specifically, state detector 420 first checks whether all of the following conditions are satisfied by values in the data point (i.e., DP2):
A. Mass-difference (MD-2) is a negative value and exceeds a pre-determined threshold
B. Depth-difference (DD-2) is a positive value (indicating an increase in depth)
C. Vertical-movement-present is ‘Y’

If all conditions noted above are satisfied, state detector 420 sends RGB-ET-2 contained in DP2 to operative model 460.

If operative model 460 infers ‘baby absent’ for RGB-ET-2, state detector 420 checks whether inner movement in cradle 200 (based on IM-2 value in DP2) is below a pre-determined lower threshold and breathing rate (based on RR-2 value in DP2) is below a pre-determined lower threshold (i.e., inner movement and breathing pattern are not detected), and if so, concludes that INV-2 corresponds to a baby retrieval activity.

When state detector 420 determines baby retrieval activity, state detector 420 records the time instance of intervention end-time (I-ET-2) of DP2 as start time of ‘baby absent’ state (BA-ST) if a previously recorded BA-ST does not exist. Additionally, state detector 420 records intervention start-time (I-ST-2) as the end time of ‘baby present’ state (BP-ET), but only if a BP-ST value exists. State detector 420 generates the tuple [BP-ST, I-ST-2, ‘baby present’].

However, if operative model 460 infers ‘baby present’ for RGB-ET-1 (i.e., inference of operative model 460 does not concur with collated sensor data in DP2), state detector 420 continues to examine data points received from block 410 for a pre-determined time interval (e.g., 4 hours from timestamp I-ET-2, ending at time instance T4).

Based on the data points, state detector 420 performs checks as follows:
1. If neither normal breathing rate nor inner movement is detected in duration (I-ET-2 to T4), state detector 420 concludes that baby is not present in the cradle in the duration as it is unlikely that the baby remains in the cradle without any detectable motion and/or breathing. In other words, state detector 420 concludes that INV-2 corresponds to baby retrieval activity. Accordingly, state detector 420 generates tuple [BP-ST, I-ST-2, ‘baby present’].
2. If a normal breathing rate is detected in all the data points corresponding to the time interval (I-ET-2 to T4), state detector 420 concludes that INV-2 does not correspond to ‘baby retrieval’ activity. It may be appreciated that such a scenario may occur when a heavy object (e.g., blanket or toy) was removed from cradle rather than the baby as part of INV-2. Accordingly, state detector 420 records I-ET-2 as BA-ST, but only if a previously recorded BA-ST value does not exist. Additionally, state detector 420 does not record BP-ET value. State detector 420 does not generate any tuple corresponding to INV-12
3. If a normal breathing rate is not detected but inner movements are detected in data points corresponding to time interval (I-ET-2 to T4), state detector 420 re-evaluates inferences received from operative model 460, as follows:
   i). If operative model 460 infers ‘baby absent’ in more than a threshold percentage (e.g., 80%) of the RGB images captured in the time interval (I-ET-2 to T4), state detector 420 concludes that INV-2 corresponds to a baby retrieval activity. State detector 420 accordingly generates the tuple [BP-ST, I-ST-2, ‘baby present’].
   ii). If operative model 460 infers ‘baby present’ in more than the threshold percentage of the RGB images captured in the time interval (I-ET-2 to T4), state detector 420 concludes that the INV-2 corresponds to a baby placement activity. Accordingly, state detector 420 records time instance I-ET-2 as BP-ST if BP-ST value does not exist. Also, state detector 420 records time instance I-ST-2 as BA-ET, but only if BA-ST value exists. State detector 420 generates tuple [BA-ST, I-ST-2, ‘baby absent’].
   iii). If neither of the above conditions (i and ii) is satisfied, state detector 420 considers the case as a ‘confusion’. In such a scenario, state detector 420 checks for previously recorded BP-ST and baby BA-ST values. If BP-ST exists, state detector 420 generates tuple [BP-ST, I-ST-2, ‘baby present’]. If BA-ST exists, state detector 420 generates tuple [BA-ST, I-ST-2, ‘baby absent’].

It may be appreciated that if state detector 420 has determined a current intervention as corresponding to a ‘baby retrieval’ activity (i.e., state detector 420 has recorded BA-ST), state detector 420 does not record an immediate subsequent intervention as baby retrieval activity even though collated sensor data and/or operative model output suggest baby retrieval. Such a scenario may occur when a baby is actually retrieved from the cradle as part of a first intervention while a heavy object (e.g., toy or blanket) mimics retrieval of a baby in an immediately subsequent intervention.

It may be appreciated that, as noted above, state detector 420 performs additional checks for existence of either a normal breathing rate or inner movements in the duration BP-ST to BP-ET in order to determine ‘baby present’ state. Such reasoning is based on the understanding that it is improbable for a baby to remain motionless for extended periods without any detectable breathing. Conversely, state detector 420 verifies absence of a detectable breathing pattern and detectable inner movement in the duration BA-ST to BA-ET in order to determine ‘baby absent’ state. It is worth noting that, as noted above, false positives for inner movement may occur even when the baby is not present in the cradle due to, for example, factors such as swaying objects hanging near the sensor(s).

The description is continued to illustrate the manner in which record generator 430 generates data records based on state indicator data received from block 420.

### 10. Record Generation

In an embodiment, record generator 430 determines time windows for labeling images as ‘baby present’ and ‘baby absent’ based on state indicators received from block 420. The time interval (BP-ST to BP-ET) of ‘baby present’ state is referred to as a ‘Baby Present’ time window. The time interval (BA-ST to BA-ET) of ‘baby absent’ state is referred to as a ‘Baby Absent’ time window.

Record generator 430 sets label ‘baby present’ for images corresponding to ‘baby present’ time window and label ‘baby absent’ for images corresponding to ‘baby absent’ time window. Thus, each data record includes an image and a corresponding label indicating whether baby is present or absent in the cradle corresponding to the image. Record generator 430 stores the labelled data records in data store 470. Record generator 430 may be implemented to generate data records periodically (e.g., as a scheduled batch job) by retrieving RGB images and state indicators from data store 470, and labeling the retrieved images.

It may be appreciated that the training and test data records are generated automatically (without human intervention) by correlating RGB image data with sensor data and output of operative model.

In each time window, record generator 430 marks a subset of images as ‘training data’ and the rest of the images in the time window as ‘test data’. As an example, if there are 10 RGB images retrieved from data store 470 corresponding to a ‘baby present’ time window, record generator may mark 6 images as ‘training data’ and the remaining 4 images as ‘test data’. A convenient subset of images in each time window may be selected for ‘training’ and ‘testing’ as will be apparent to a skilled practitioner by reading the disclosure herein.

The description is continued to illustrate some example scenarios of state detection and record generation according to aspects of the present disclosure.

### 11. Example State Detection and Record Generation

FIG. 5B is a timing diagram (not to scale) illustrating the manner in which state detector 420 operates to determine ‘baby present and ‘baby absent’ states based on collated sensor data and intervention data received from data collation block 550, and inference of operative model 460. The Figure depicts example interventions IV-1 (555-1) through IV-4 (555-4) with corresponding start-times and end-times. Thus, IV-1 is shown to start at time instance t511 and end at time instance t512. Similarly, start-times/end-times of IV-2, IV-3 and IV-4 are respectively t521/t522, t531/t532 and t541/542. Time instances t510, t520, t530 and t540 respectively depict the time instances just prior to corresponding intervention start-times.

It is assumed that prior to t510 (e.g., 2 hours prior to t510), baby is not present in cradle 200, and state detector 420 accordingly has recorded a BA-ST value (tx1) from a previous intervention (not shown in FIG. 5B). Also, state detector 420 has not recorded a BP-ST value prior to t510.

At t511, intervention IV-1 starts. Assuming that baby is being placed in cradle 200, movement monitor 510 detects boundary movement lasting for more than 3 seconds. Boundary movement persists till t512. Accordingly, movement monitor 510 records intervention start-time as t511 and end-time as t512 for IV-1.

At t512, data collation block receives intervention start-time (t511) and end-time (t512) from block 510, and operates to calculate difference in mass and depth respectively by comparing data received from distance sensor (214) and ToF sensor at time instances t510 and t512. Data collation block 550 determines that vertical movement is present in the intervention duration by comparing accelerometer (204) data at time instances t510 and t512. Data collation block 550 receives RGB images captured at t511 and t512 from image capture block 540.

At t512, state detector 420 receives data point DP1 containing values [t511, t512, md-1, dd-1, ‘Y’, im-1, rr-1, RGB-t511, RGB-t512] from data analyzer 410. State detector 420 determines that the following conditions are satisfied:
A. Mass difference (md-1) is a positive value and exceeds a pre-determined threshold
B. Depth difference (dd-1) is a negative value (indicating a decrease in depth)
C. Vertical-movement-present is ‘Y’

State detector 420 sends RGB-t512 contained in DP1 to operative model 460. It is assumed that operative model 460 infers ‘baby present’ for the RGB image. State detector 420 accordingly generates tuple [tx1, t511, ‘baby absent’]. State detector 420 also records t512 as BP-ST.

At t521, intervention IV-2 starts. Assuming that baby is being retrieved from cradle 200, movement monitor 510 detects boundary movement lasting for more than 3 seconds. Boundary movement persists till t522. Accordingly, movement monitor 510 generates intervention start-time as t521 and end-time as t522 for IV-1.

At t522, data collation block receives intervention start-time (t521) and end-time (t522) from block 510, and operates to generate data point DP2 based on sensor data obtained at t520 (just prior to start of intervention IV-2) and t522 (end of intervention IV-2).

At t522, state detector 420 receives data point DP2 containing values [t521, t522, md-2, dd-2, ‘Y’, im-2, rr-2, RGB-t521, RGB-t522] from data analyzer 410. State detector 420 determines that the following conditions are satisfied:
A. Mass difference (md-2) is a negative value and exceeds a pre-determined threshold
B. Depth difference (dd-2) is a positive value (indicating an increase in depth)
C. Vertical-movement-present is ‘Y’

State detector 420 sends RGB-t522 contained in DP2 to operative model 460. It is assumed that operative model 460 infers ‘baby absent’ for the RGB image. State detector 420 accordingly generates tuple [t512, t521, ‘baby present’]. State detector 420 also records t522 as BA-ST.

At t531, intervention IV-3 starts. Assuming that a heavy blanket (equivalent to weight of baby) is being placed in cradle 200, movement monitor 510 detects boundary movement lasting for more than 3 seconds. Boundary movement persists till t532. Accordingly, movement monitor 510 generates intervention start-time as t531 and end-time as t532 for IV-3.

At t532, state detector 420 receives data point DP3 containing values [t531, t532, mm-3, dd-3, ‘Y’, im-3, rr-3, RGB-t531, RGB-t532] from data analyzer 410. State detector 420 determines that the following conditions are satisfied:
A. Mass difference (md-3) is a positive value and exceeds a pre-determined threshold
B. Depth difference (dd-3) is a negative value (indicating a decrease in depth)
C. Vertical-movement-present is ‘Y’

State detector 420 sends RGB-t532 contained in DP1 to operative model 460. It is assumed that operative model 460 infers ‘baby absent’ for the RGB image. State detector 420 therefore does not generate any tuple corresponding to IV-3 but continues to examine data points received from block 410 in the time duration t532-t538.

State detector 420 performs the additional checks noted above, and determines that neither inner movement threshold nor respiratory rate threshold is exceeded. Accordingly, state detector 420 does not generate any tuple corresponding to IV-3. State detector 420 does not record t532 as BA-ST since a previous value (t522) exists for BA-ST.

At t541, intervention IV-4 starts. Assuming that baby is being placed in cradle 200, movement monitor 510 detects boundary movement lasting for more than 3 seconds. Boundary movement persists till t542. Accordingly, movement monitor 510 generates intervention start-time as t541 and end-time as t542 for IV-1.

At t542, state detector 420 receives data point DP4 containing values [t541, t542, md-4, dd-4, ‘Y’, im-4, rr-4, RGB-t541, RGB-t542] from data analyzer 410.

State detector 420 determines that conditions A-C are satisfied, and sends RGB-t512 contained in DP4 to operative model 460. It is assumed that operative model 460 infers ‘baby present’ for the RGB image. State detector 420 accordingly generates tuple [t522, t541, ‘baby absent’]. State detector 420 also records t541 as BP-ST.

Based on the above tuples generated by state detector 420, record generator 430 operates to generate data records. Specifically, record generator 430 receives the following tuples corresponding to the interventions depicted in FIG. 5A:
[tx1, t511, ‘baby absent’].
[t512, t521, ‘baby present’]
[t522, t541, ‘baby absent’].

Accordingly, record generator sets labels as follows for the RGB images captured in corresponding time intervals
tx1 to t511-‘Baby absent’
t512 to t521-‘Baby present’
t522 to t541-‘Baby absent’

It may be appreciated that although intervention IV-3 mimics a baby placement activity (due to placing of a heavy blanket in the cradle), aspects of the present disclosure operate to avoid incorrectly labeling RGB images in the time interval t532-t541 as ‘baby present’.

Data records thus generated are used by model manager 450 in order to evaluate the teacher model and the student model and/or fine-tune the student model. Accordingly, the description is continued to illustrate the implementation details of a model manager in an embodiment of the present disclosure.

### 12. Model Manager

FIG. 5C is a block diagram illustrating an example implementation of a model manager (450) in an embodiment of the present disclosure. The block diagram is shown containing models 560 (in turn shown containing teacher model 565, student model 570 and fine-tuned student model 570-F), model selector 575, model trainer 580 and model forwarder 585.

Models 560 represents ML models used for inferring whether a baby is present or absent in a cradle, given an RGB image as input. Models 565 and 570 respectively represent model information of a teacher model and a student model received from central model controller 170. Model 570-F represents a fine-tuned version of student model 570.

Model selector 575 retrieves (via path 121) a teacher model and a student model from central model controller 170, and labelled data records from data store 470 (via path 453). Model selector 575 saves the model information of the received teacher model and student model as model 565 and 570 respectively. Model selector 575 operates to select one of models 560 as operative model 460.

In an embodiment, model selector 575 places request to central model controller 170 for models 560 and 570 only when the number of training data records and test data records in data store 470 exceed respective pre-determined threshold counts. Alternatively, model selector 575 may place the request for the models periodically at preset time points (e.g., once every 15 days). As an example, model manager 575 may place the request when at least 150 ‘training’ images and 40 ‘test’ images are determined to be present for each of ‘baby present’ and ‘baby absent’ labels in data store 470. However, other suitable counts of training and test data records may be selected based on specific implementation of BMS 150, as will be apparent to a skilled practitioner by reading the disclosure herein.

According to an aspect of the present disclosure, upon receipt of the teacher model and the student model, model selector 575 retrieves data records from data store 470, and feeds each image of each data record to teacher model 565 to cause teacher model 565 to infer whether baby is present or absent in the cradle. Model selector 575 checks whether each inference of teacher model 565 matches the corresponding label of the same record as a basis to determine whether teacher model 565 infers with a desired accuracy (hereafter TM-accuracy). Model selector 575 checks accuracy of teacher model 565 using training data records and/or test data records. If the accuracy of teacher model 565 is equal to or greater than TM-accuracy, model selector 575 selects teacher model 565 as operative model 460. In an embodiment, TM-accuracy equals 95%.

If the accuracy of teacher model 565 is less than TM-accuracy, model selector 575 indicates to model trainer 580 to fine-tune student model 570. Upon receiving a notification from model trainer 580 that the student model has been fine-tuned, model selector 575 checks the accuracy of fine-tuned student model 570-F. Specifically, model selector 575 feeds each image of each ‘test’ data record to model 570-F to cause the model to infer whether baby is present or absent in said cradle. Model selector 575 checks whether each inference of fine-tuned student model 570-F matches the corresponding label of the same record as a basis to determine whether model 570-F infers with a desired accuracy (hereafter SM-accuracy). If the accuracy of model 570-F is equal to or greater than SM-accuracy, model trainer 580 selects student model 570-F as operative model 460. In an embodiment, SM-accuracy equals 95%.

Alternatively, the performance of model 570-F may be evaluated using a threshold-based approach that considers accuracy of both student model 470 (prior to fine-tuning) and model 570-F (post fine-tuning). Such an approach is described below using example values for convenience:
1. Low Accuracy Baseline: If accuracy of model 570 is less than 75%, then model 570-F is deemed to perform well if accuracy of 570-F is equal to or greater than 80%.
2. Moderate Accuracy Baseline: If accuracy of model 570 is 75%-85%, then model 570-F is deemed to perform well if accuracy of 570-F is equal to or greater than 90%.
3. High Accuracy Baseline: If accuracy of model 570 is greater than 90%, then model 570-F is deemed to perform well if accuracy of 570-F is equal to or greater than 95%.

If model 570-F is determined to perform well based on above-noted criteria, model selector 575 selects student model 570-F as operative model 460. If model 570-F is determined to not perform well, model selector 575 indicates to model trainer 580 that the student model needs to be fine-tuned further. Thus, the steps of checking accuracy of fine-tuned student model and fine-tuning are performed iteratively.

After selecting the operative model, model selector 575 sends training and test data records to central model controller 170, and thereafter deletes data records in data store 470. Model selector 575 sends data records (RGB images along with class labels) to block 170 only if the end-user (e.g., parents/care-giver of baby) of BMS 150 permits to do so. Such permission information may be configured in and read from data store 470 in a known way. If model selector 575 selects fine-tuned model 570-F as the operative model, model selector sends model information of fine-tuned model 570-F to model forwarder 585.

It is noted herein that when updating the operative model with either the teacher or fine-tuned student model, model selector 575 loads the selected model for inferences, for example, upon system restart. When the baby is not present in the cradle, the service is restarted automatically, ensuring a seamless transition to the selected model.

Model forwarder 585 forwards (via path 121) the model information received from model selector 575 to central model controller 170.

Model trainer 580 receives an indication from model selector 575, and operates to fine-tune the student model in response to such indication. Model trainer 580 checks if a previously saved model state (of model 570-F) exists in models 560. If no such saved state exists, model trainer 580 operates to fine-tune student model 570 to generate fine-tuned student model 570-F. If a saved state exists in models 560, model trainer 580 operates to resume fine-tuning from the previously saved state of model 570-F. Model trainer 580 sets fine-tuning parameters such as learning rate, batch size, number of training epochs, optimizer type, weight decay, early stopping criteria, and regularization techniques, etc. as is well known in the relevant arts. Model trainer 580 may set such parameters based on prior configuration information retrieved (via path 453) from data store 470.

According to an aspect, model trainer 580 fine-tunes the student model over one or more training sessions. Each ‘training session’ of the fine-tuning process refers to training the student model for a pre-determined number of training epochs. As is well known in the relevant arts, an epoch refers to one complete pass of a training dataset through a learning algorithm. At the start of each training session, model trainer 580 retrieves data records marked as ‘training data’ from data store 470. At the end of each training session, model trainer 580 saves the resulting state (such as the hyperparameters, weights, biases, etc.) of model 570-F in models 560, and notifies model selector 575 that the student model has been fine-tuned. Model trainer 580 may omit certain training data records from being used for fine-tuning the student model if images in the data records exhibit significant similarity. The similarity may be evaluated in a known way. In an embodiment, cosine similarity function is used to determine similarity between images.

In an embodiment, model trainer 580 waits for a pre-determined time duration (e.g., 24 hours) to elapse between training sessions. In the wait duration, additional data records are generated which are augmented to the training data used to fine-tune the student model in the subsequent training session. Model trainer 580 performs the fine-tuning only when the baby is not present in the cradle (as indicated by state detector 420).

In an embodiment, the number of epochs is 10. In general, the fine-tuning parameters are configured to prevent or at least minimize underfitting or overfitting the student model, as will be apparent to a skilled practitioner.

It is noted herein that although the illustrative embodiment describes using ‘accuracy’ as a measure for evaluating performance of models, alternative measures such as speed of inference, scalability, precision, recall, F1 score or a combination of these measures may be used as the basis of selecting the best/suitable model as the operative model.

The description is continued to illustrate an example implementation of a central model controller according to aspects of the present disclosure.

### 13. Central Model Controller

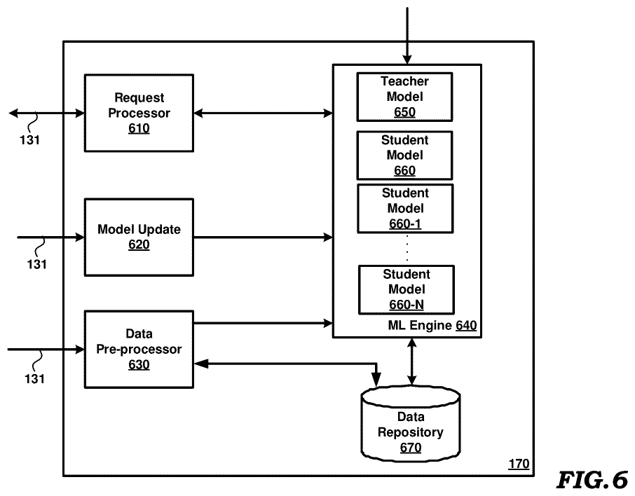

FIG. 6 is a block diagram illustrating the implementation details of a central model controller (170) in an embodiment of the present disclosure. The block diagram is shown containing request processor 610, model update 620, data pre-processor 630, ML engine 640 and data repository 670. Each of the blocks in the Figure is described in detail below.

Data repository 670 represents a non-volatile (persistent) storage facilitating storage and retrieval of data by model update 620, data pre-processor 630 and ML engine 640. In particular, data repository 620 maintains data records received from various BMSs 150. Data repository 670 may be implemented as a database server using relational database technologies and accordingly provide storage and retrieval of data using structured queries such as SQL (Structured Query Language). Alternatively or in addition, data repository 670 may be implemented as a file server providing storage and retrieval of data in the form of files organized as one or more directories, as is well-known in the relevant arts.

Request processor 610 receives (via path 131) requests for models from BMS 150. Each request from BMS 150 may contain a unique identifier of the corresponding BMS from which the request originates. Request processor 610 forwards the request to ML engine 640, receives the model information of teacher model 650 and student model (660 or a corresponding one of 660-1 to 660-N), and forwards the model information to the requesting BMS on path 131.

Model update 620 receives fine-tuned student model information (via path 131) from BMS 150 and forwards the model information to ML engine 640.

Data pre-processor 310 receives data records from BMS 150, performs pre-processing on the data records and stores the processed data records in data repository 670. Pre-processing includes but is not limited to removing incorrect/incomplete data, discretization of data, normalization of data, identification of feature set, etc. as is well known in the relevant arts.

ML engine 640 generates and maintains teacher model 650, base student model 660 and BMS-specific fine-tuned student models 660-1 to 660-N. Teacher model 650 represents a global model shared across all BMS 150. Base student model 660 represents a pre-trained student model 660 formed/generated in central model controller 170. The teacher model may be generated using any machine learning approach such as KNN (K Nearest Neighbor), Decision Tree, etc. Various other machine learning approaches can be employed, as will be apparent to skilled practitioners, by reading the disclosure provided herein.

In an embodiment, supervised machine learning approaches are used to form the teacher model, and knowledge distillation techniques are used to form the base student model. The manner in which ML engine 640 generates the teacher model and the base student model is described below with examples. ML engine 640 may fine-tune teacher model 650 periodically.

When a request for models is received from request processor 610, ML engine checks if a fine-tuned model (one of 660-1 to 660-N) for the requesting BMS exists. If so, ML engine sends the model information to block 610. If no fine-tuned model exists for the requesting BMS, ML engine sends the model information of base student model 660 to block 610.

When a model update is received from block 620, ML engine 640 replaces the corresponding fine-tuned student model information with the received model information.

The description is continued to illustrate the manner in which teacher model and student model are formed according to aspects of the present disclosure.

### 14. Forming the Teacher Model and the Base Student Model

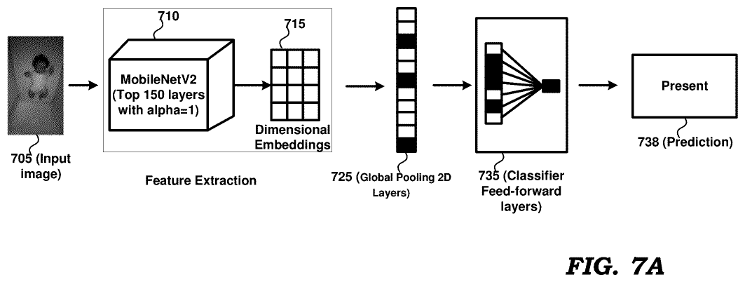

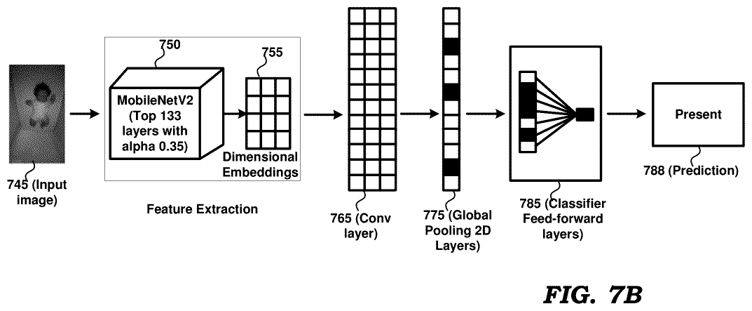

FIGS. 7A and 7B respectively depict the various stages of forming teacher model 650 and base student model 660 in an embodiment of the present disclosure. In the illustrative embodiment, MobileNetv2 architecture from Google® is employed to form the models. Specifically, as depicted in FIG. 7A, data pre-processor 630 pre-processes input RGB image (705) and obtains embeddings of the input (715) from MobileNetv2 (710), resulting in an image embedding of dimensions (8*15*1280). Subsequently, ML engine 640 applies GlobalPooling2D layer (725), resulting in an output dimension of 1280, followed by a classifier (735) with around 82,000 trainable parameters, yielding an output dimension of 1. The classifier serves the purpose of categorizing the input image as either ‘baby present’ or ‘baby absent’ (738, prediction). The teacher model may optionally be fine-tuned in a known way.

FIG. 7B depicts the manner in which base student model 660 is formed from teacher model 650 using knowledge distillation. Specifically, the student model is trained using the same training data as used for the teacher model. Feature embeddings of the input image (745) are extracted from the lightweight version (750) (e.g., alpha (width multiplier)=0.35) of MobileNetv2 model. Data pre-processor 630 employs only top 133 layers of MobileNetv2 to obtain the feature embedding of the input (as compared to employing 150 layers when forming the teacher model), resulting in an image embedding (755) of dimensions (8*15*56). Subsequently, ML engine 640 feeds the feature embedding into a conv-2d layer (765), generating an output with dimensions (8*15*1280). Following this, a GlobalPooling2D layer (775) and a classifier (785) are applied, mirroring the architecture of the teacher model, ultimately resulting in an output dimension of 1. The classifier serves the purpose of categorizing the input image as either ‘baby present’ or ‘baby absent’ (788, prediction). The trainable parameters selected for the student model are the same as those selected for the teacher model. In general, parameters for the student model may be configured so as to obtain an optimal trade-off between complexity and accuracy of the student model, as will be apparent to a skilled practitioner.

It may be appreciated that in the process of knowledge distillation, the output of the global pooling layer (referred to as GlobalPooling2D in FIGS. 7A and 7B), serves as a feature representation for both the teacher and student models. Thus, feature-based knowledge distillation is employed to seamlessly transfer the acquired knowledge from the teacher to the student model.

The description is continued to illustrate the manner in which presence or absence of babies in cradles is detected using baby monitoring systems.

### 15. Baby Detection

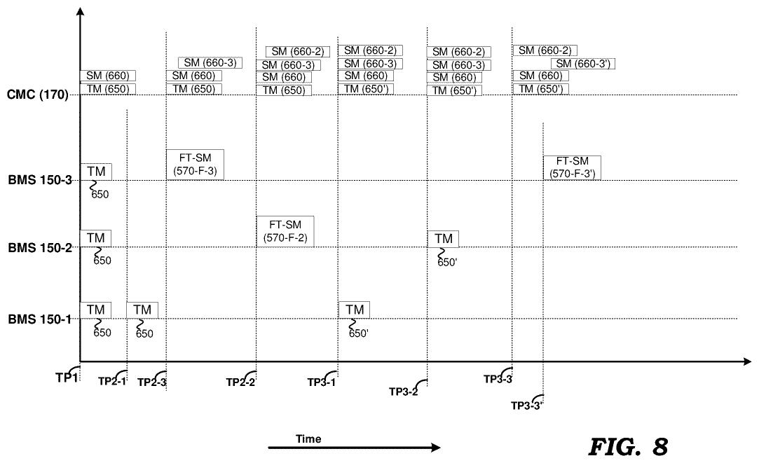

FIG. 8 is a timing diagram illustrating various example scenarios of baby detection according to aspects of the present disclosure. The Figure depicts the operative model at various points in time in three baby monitoring devices-BMS 150-1, 150-2 and 150-3, and models stored in central model controller 170. Reference is made to blocks of FIGS. 4, 5A, 5C and 6 as appropriate.

A. Installation of a New Baby Monitoring System with Associated Cradle

Prior to time point TP1, it is assumed that teacher model 650 and base student model 660 are stored in central model controller 170.

At TP1, upfront installation of BMS 150-1, 150-2 and 150-3 occurs at respective cradles. Merely for ease of illustration, it is assumed that upfront installation of BMS 150-1 to 150-3 occurs at a same time point (i.e., TP1). Accordingly, each of BMS 150-1, 150-2 and 150-3 receives the global (upfront) teacher model (650) at TP1. The teacher model is selected as the operative model 460 at each of BMS 150-1 to 150-3. Each BMS 150 uses steps 310-380 in the flow-chart of FIG. 3 for baby detection.

Specifically, in step 310, in a first duration following the upfront installation, each of BMS 150-1, 150-2 and 150-3 generates data records local to the respective cradle. Since the usage of each cradle may vary, data records generated in step 310 may exceed the pre-determined threshold count noted above at different time points. Accordingly, as depicted in the Figure, BMS 150-1, 150-2 and 150-3 retrieve the teacher model and the student model from central model controller 170 at TP2-1, TP2-2 and TP2-3 respectively, with TP2-2 being subsequent in time with respect to TP2-3. The description is continued to illustrate various example scenarios for baby detection.

### B. Continuing to Use the Teacher Model

In an example scenario, at TP2-1, upon receiving a request for models from BMS 150-1, ML engine 640 checks if a fine-tuned model (i.e., 660-1) exists for BMS 150-1. Since no fine-tuned student model has been generated yet by BMS 150-1, ML engine 640 sends the model information of base student model 660 (and teacher model 650) to BMS 150-1. Model selector 575-1 (FIG. 5C) saves the model information of the received teacher model and student model as model 565-1 and 570-1 respectively.

In step 320, model manager 450-1 (FIG. 4) feeds each image of each data record to the teacher model, and in step 330, checks whether inferences of the teacher model match the corresponding labels with a first desired accuracy. It is assumed that the first desired accuracy is satisfied by the teacher model for BMS 150-1. Accordingly, model manager 450-1 continues to use the teacher model as the operative model (step 340).

C. Replacing the Teacher Model with a Fine-tuned Student Model

In another example scenario, at TP2-3, upon receiving a request for models from BMS 150-3, ML engine 640 checks if a fine-tuned model (i.e., 660-3) exists for BMS 150-3. Since no fine-tuned student model has been generated at BMS 150-3, ML engine 640 sends the model information of base student model 660 to BMS 150-3. Model selector 575-3 saves the model information of the received teacher model and student model as model 565-3 and 570-3 respectively.

In step 320, model selector 575-3 feeds each image of each data record to the teacher model, and in step 330, checks whether inferences of the teacher model match the corresponding labels with a first desired accuracy. It is assumed that the first desired accuracy is not satisfied by the teacher model for BMS 150-3. Accordingly, model manager 450-3 (more specifically, model trainer 580-3) fine-tunes (in step 350) the base student model (570-3) to generate model 570-F-3.

In step 360, model selector 575-3 feeds each image of each data record to the fine-tuned student model (FT-SM, 570-F-3), and in step 370, checks whether inferences of the fine-tuned student model match the corresponding labels with a second desired accuracy. It is assumed that the second desired accuracy is satisfied by model 570-F-3. Accordingly, model selector 575-3 uses the fine-tuned student model as the operative model (step 380). Model forwarder 585-3 forwards the model information of the fine-tuned student model to central model controller 170. ML engine 640 stores the model information as model 660-3 (corresponding to BMS 150-3), as depicted in the Figure.

It is assumed that at TP2-2, BMS 150-2 operates in a manner similar to BMS 150-3, and accordingly generates a fine-tuned student model FT-SM, 570-F-2, the information of which is stored as model 660-2 by ML engine 640.

After updating the respective operative model, the data records generated in step 310 by each of BMS 150-1, 150-2 and 150-3 are sent to central model controller 170, and subsequently deleted in respective data stores 470.

It may be appreciated that the teacher model may fail to perform baby detection with the first desired accuracy in certain scenarios, particularly when additional objects such as printed bedsheets, soft toys (e.g., dolls), heavy blankets, hanging kind of stuff, or printed pillows are present in the cradle. Such items can occasionally lead to incorrect detections by mimicking features associated with a baby. By collecting data from the specific cradle environment, the student model may be fine-tuned to learn the unique characteristics of the cradle and distinguish between features associated with the baby and those of the environment. The fine-tuned student model may thus meet the desired second accuracy, making the model better suited for accurate baby detection compared to the teacher model.

At the end of a second duration following the first duration, BMS 150-1, 150-2 and 150-3 retrieve the teacher model and the student model from central model controller 170 at TP3-1, TP3-2 and TP3-3 respectively (step 315). In the time duration spanning TP2-2 and TP3-1, it is assumed that the teacher model is fine-tuned by central model controller 170 to generate teacher model 650′.

At TP3-1, BMS 150-1 executes steps 320 and 330. It is assumed that the first desired accuracy is satisfied by teacher model (650′/565) for BMS 150-1. Accordingly, model manager 450-1 continues to use the teacher model as the operative model (step 340).

D. Replacing the Fine-Tuned Student Model with the Teacher Model

In another example scenario, at TP3-2, BMS 150-2 executes steps 320 and 330. It is assumed that first desired accuracy is satisfied by teacher model (650′) for BMS 150-2. Accordingly, model selector 575-2 updates the operative model to the teacher model without checking whether the second desired accuracy is satisfied by model 570 (660-2).

It may be appreciated that babies experience developmental and behavioral changes over time, which may coincide with alterations in the cradle environment. As a result, the teacher model, which may have previously not satisfied the first desired accuracy for a specific cradle, might begin to perform well because the environmental factors—such as bedding conditions, toys, or other elements—have become more aligned with learned knowledge of the teacher model.

The teacher model, having been trained and fine-tuned using data from multiple cradles, may have a broader understanding and learns more generalized features than a student model.

E. Replacing the Current Fine-Tuned Student Model with a Recent Fine-Tuned Model

In another example scenario, at TP3-3, BMS 150-3 executes steps 320 and 330. It is assumed that first desired accuracy is not satisfied by teacher model (650′) for BMS 150-3. Accordingly, model selector 575-3 checks if the student model (570/660-3) meets the second desired accuracy. Such check is performed by BMS 150-3 using test data records generated in duration (TP2-3 to TP3-3). It is assumed that second desired accuracy is not satisfied by the student model. Accordingly, model trainer 580-3 fine-tunes model 570 (660-3) to generate 570-F-3′ using training data records generated in duration (TP2-3 to TP3-3). As noted above, model trainer 580-3 fine-tunes the student model (over multiple training sessions) until the fine-tuned student model infers with the second desired accuracy at TP3-3′. Alternatively, the baseline approach may be employed to determine whether the fine-tuned student model is inferring with the desired accuracy or not.

At TP3-3′, model selector 575-3 thereafter uses model 570-F-3′ as operative model 460. Model forwarder 585-3 forwards the model information of the fine-tuned student model (570-F-3′) to central model controller 170. ML engine 640 replaces model 660-3 (corresponding to BMS 150-2) with 660-3′. Data records generated in duration (TP2-3 to TP3-3) are deleted.

In this manner, aspects of the present disclosure enable detecting presence or absence of babies in cradles using monitoring systems.

It is noted herein that merely as an illustration, the features are described as being performed in an edge device. However, some of the features can be performed in other digital processing systems, as will be apparent to a skilled practitioner.

Similarly, while the techniques are descried with respect to a baby in a cradle, the features can be implemented with respect to any sleeping surfaces as well.

In general, the techniques described herein can be applied in other scenarios/environments having similar requirements and/or complexities, as will be apparent to a skilled practitioner by reading the disclosure herein.

The description is continued with respect to an embodiment in which various features are operative when the software instructions described above are executed.

### 16. Digital Processing System

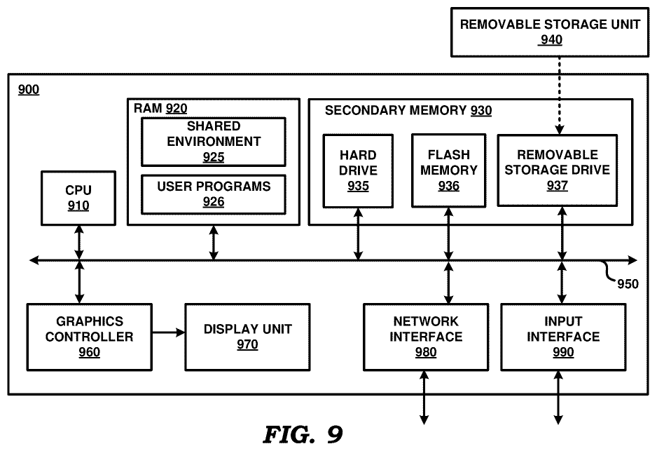

FIG. 9 is a block diagram illustrating the details of digital processing system 900 in which various aspects of the present disclosure are operative by execution of appropriate executable modules. Digital processing system 900 may correspond to baby monitoring system 150 and central model controller 170 (or any system implementing baby monitoring system 150/central model controller 170).

Digital processing system 900 may contain one or more processors such as a central processing unit (CPU) 910, random access memory (RAM) 920, secondary memory 930, graphics controller 960, display unit 970, network interface 980, and input interface 990. All the components except display unit 970 may communicate with each other over communication path 950, which may contain several buses as is well known in the relevant arts. The components of FIG. 9 are described below in further detail.

CPU 910 (including any specific hardware specific to machine learning, etc.) may execute instructions stored in RAM 920 to provide several features of the present disclosure. CPU 910 may contain multiple processing units, with each processing unit potentially being designed for a specific task. Alternatively, CPU 910 may contain only a single general-purpose processing unit.

RAM 920 may receive instructions from secondary memory 930 using communication path 950. RAM 920 is shown currently containing software instructions constituting shared environment 925 and/or other user programs 926 (such as other applications, DBMS, etc.). In addition to shared environment 925, RAM 920 may contain other software programs such as device drivers, virtual machines, etc., which provide a (common) run time environment for execution of other/user programs.

Graphics controller 960 generates display signals (e.g., in RGB format) to display unit 970 based on data/instructions received from CPU 910. Display unit 970 contains a display screen to display the images defined by the display signals. Input interface 990 may correspond to a keyboard and a pointing device (e.g., touch-pad, mouse) and may be used to provide inputs. Network interface 980 provides connectivity to a network (e.g., using Internet Protocol), and may be used to communicate with other systems connected to the networks.

Secondary memory 930 may contain hard drive 935, flash memory 936, and removable storage drive 937. Secondary memory 930 may store the data (e.g., data portions of FIGS. 4 and 6) and software instructions (e.g., for implementing the steps of FIG. 3, for implementing the blocks of FIGS. 4, 5A, 5C and 6), which enable digital processing system 900 to provide several features in accordance with the present disclosure. The code/instructions stored in secondary memory 930 may either be copied to RAM 920 prior to execution by CPU 910 for higher execution speeds, or may be directly executed by CPU 910.

Some or all of the data and instructions may be provided on removable storage unit 940, and the data and instructions may be read and provided by removable storage drive 937 to CPU 910. Removable storage unit 940 may be implemented using medium and storage format compatible with removable storage drive 937 such that removable storage drive 937 can read the data and instructions. Thus, removable storage unit 940 includes a computer readable (storage) medium having stored therein computer software and/or data. However, the computer (or machine, in general) readable medium can be in other forms (e.g., non-removable, random access, etc.).

In this document, the term “computer program product” is used to generally refer to removable storage unit 940 or hard disk installed in hard drive 935. These computer program products are means for providing software to digital processing system 900. CPU 910 may retrieve the software instructions, and execute the instructions to provide various features of the present disclosure described above.

The term “storage media/medium” as used herein refers to any non-transitory media that store data and/or instructions that cause a machine to operate in a specific fashion. Such storage media may comprise non-volatile media and/or volatile media. Non-volatile media includes, for example, optical disks, magnetic disks, or solid-state drives, such as storage memory 930. Volatile media includes dynamic memory, such as RAM 920. Common forms of storage media include, for example, a floppy disk, a flexible disk, hard disk, solid-state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge.

Storage media is distinct from but may be used in conjunction with transmission media. Transmission media participates in transferring information between storage media. For example, transmission media includes coaxial cables, copper wire and fiber optics, including the wires that comprise bus 950. Transmission media can also take the form of acoustic or light waves, such as those generated during radio-wave and infra-red data communications.

Reference throughout this specification to “one embodiment”, “an embodiment”, or similar language means that a particular feature, structure, or characteristic described in connection with the embodiment is included in at least one embodiment of the present disclosure. Thus, appearances of the phrases “in one embodiment”, “in an embodiment” and similar language throughout this specification may, but do not necessarily, all refer to the same embodiment.

Furthermore, the described features, structures, or characteristics of the disclosure may be combined in any suitable manner in one or more embodiments. In the above description, numerous specific details are provided such as examples of programming, software modules, user selections, network transactions, database queries, database structures, hardware modules, hardware circuits, hardware chips, etc., to provide a thorough understanding of embodiments of the disclosure.

### 17. Conclusion

References throughout this specification to “one embodiment”, “an embodiment”, or similar language means that a particular feature, structure, or characteristic described in connection with the embodiment is included in at least one embodiment of the present invention. Thus, appearances of the phrases “in one embodiment”, “in an embodiment” and similar language throughout this specification may, but do not necessarily, all refer to the same embodiment. The following examples pertain to above or further embodiments.

While various embodiments of the present invention have been described above, it should be understood that they have been presented by way of example only, and not limitation. Thus, the breadth and scope of the present invention should not be limited by any of the above-described embodiments, but should be defined only in accordance with the following claims and their equivalents.

What is claimed is:

1. A method performed in a digital processing system for detecting presence or absence of a baby in a cradle, said digital processing system being deployed associated with said cradle, said method comprising:
generating a first plurality of data records with each data record having an image and a corresponding label indicating whether baby is present or absent in said cradle corresponding to the image;
feeding each image of said first plurality of data records to a first teacher model to cause said first teacher model to infer whether baby is present or absent in said cradle;
checking whether said inferences of said first teacher model match said corresponding labels with a first desired accuracy;
if yes: continuing to use said first teacher model as an operative model to thereafter infer whether baby is present or absent based on corresponding received images;
if no: using a first student model as said operative model thereafter to infer whether baby is present or not based on corresponding received images,
wherein said first student model is formed by knowledge distillation from said teacher model.

2. The method of claim 1, wherein said generating is performed using an upfront teacher model as said operative model when said digital processing system is deployed upfront upon installation of said cradle.

3. The method of claim 1, further comprising fine-tuning said first student model using a first set of training records prior to using said first student model as said operative model, wherein each training record comprises an image and a corresponding label indicating whether baby is present or absent in said cradle corresponding to the image.

4. The method of claim 3, wherein said first teacher model and said first student model are received after said generating, wherein said first teacher model does not infer with said first desired accuracy,
wherein said first set of training records corresponding to a first subset of said first plurality of data records in performing said fine-tuning of said first student model, wherein said fine-tuning is performed upon said checking determining that said first teacher model does not infer with said first desired accuracy,
said method further comprising:
feeding each image of a second subset of said first plurality of data records to said fine-tuned first student model to cause said fine-tuned first student model to infer whether baby is present or absent in said cradle;
checking whether said inferences of said fine-tuned first student model match said corresponding labels in said second subset with a second desired accuracy;
if yes, using said fine-tuned first student model as said operative model to thereafter infer whether baby is present or absent based on corresponding received images.

5. The method of claim 4, wherein said operative model is used for inferring in a first duration following said upfront installation, said method further comprising:
generating a second plurality of data records after said first duration, with each data record having an image and a corresponding label indicating whether baby is present or absent in said cradle corresponding to the image;
retrieving a second teacher model and a second student model after said first duration;
feeding each image of said second plurality of data records to said second teacher model to cause said second teacher model to infer whether baby is present or absent in said cradle;
checking whether said inferences of said second teacher model for said second plurality of data records match said corresponding labels with said first desired accuracy;
if yes, using said second teacher model as said operative model to thereafter infer whether baby is present or absent based on corresponding received images.

6. The method of claim 5, wherein said second teacher model does not infer with said first desired accuracy, said method further comprising:
checking whether said second student model infers with said second desired accuracy;
if no, fine-tuning said second student model with a third subset of said second plurality of data records until said second student model infers with said second desired accuracy; and
using said fine-tuned second student model as said operative model to thereafter infer whether baby is present or absent based on corresponding received images.

7. The method of claim 6, wherein said first teacher model is fine-tuned at said central server to generate said second teacher model,
wherein said first teacher model is shared by a plurality of said digital processing systems prior to said first duration and said second teacher model is shared by said plurality of digital processing systems after said first duration,
wherein each digital processing system of a plurality of digital processing systems has a respective associated fine-tuned model stored at said central server, wherein said second student model is the corresponding fine-tune model for said digital processing system stored at said central server.

8. The method of claim 3, wherein said generating comprises:
determining the occurrence of an intervention at said cradle, wherein said intervention is characterized by an intervention-start-time and an intervention-end-time respectively corresponding to the start time instance and end time instance of said intervention, wherein an intervention duration is a time interval bounded by said intervention-start-time and said intervention-end-time;
receiving data from a plurality of sensors associated with said cradle in said intervention duration and prior to said intervention-start-time;
capturing an image of said cradle using a first camera towards said intervention-end-time;
feeding said image to said operative model to cause said operative model to generate a first inference, wherein said first inference indicates whether baby is present or absent in said cradle;
identifying said intervention as corresponding to a baby placement activity or a baby retrieval activity based on said data received from said plurality of sensors and said first inference,
wherein if said intervention is identified as corresponding to a baby placement activity:
recording said intervention-start-time as an end time of a ‘baby absent’ state and said intervention-end-time as a start time of a ‘baby present’ state,
wherein if said intervention is identified as corresponding to a baby retrieval activity:
recording said intervention-start-time as an end time of said ‘baby present’ state and said intervention-end-time as a start time of said ‘baby absent’ state;
labeling a first plurality of images captured using said first camera in a first label-duration bounded by said start time and said end time of said ‘baby present’ state with a label indicating that baby is present in said cradle; and
labeling a second plurality of images captured using said first camera in a second label-duration bounded by said start time and said end time of said ‘baby absent’ state with a label indicating that baby is absent in said cradle,
storing each image of said first plurality of images and said second plurality of images and said respective label as a corresponding data record in a data store on said digital processing system,
wherein said labeling is performed automatically.

9. The method of claim 8, wherein said plurality of sensors includes:
a second camera operable to capture 2D images and 3D images of said cradle, wherein said 3D images include depth information obtained from a time-of-flight sensor associated with said second camera;
an accelerometer operable to sense motion information inside said cradle and generate an output corresponding to said motion information;
a distance sensor operable to capture a distance from said sensor to a surface on which said cradle is kept, wherein a mass of said cradle is derived from said distance,
wherein a magnitude of inner movements in a duration of interest is determined based on 3D images captured using said second camera,
wherein a respiratory rate in said duration of interest is calculated by analyzing 3D images captured using said second camera and video captured using said first camera in said duration of interest,
wherein said identifying said intervention comprises:
calculating a mass variation value by subtracting a first mass of said cradle prior to said intervention-start-time from a second mass of said cradle at said intervention-end-time;
calculating a depth variation value by subtracting coordinates and intensity values in a first 3D image captured prior to said intervention start-time from those in a second 3D image captured at said intervention-end-time; and
determining whether vertical movement of said cradle is present or not in said intervention duration based on said motion information,
wherein said intervention is identified as corresponding to baby placement activity if:
said mass variation value is positive and exceeds a first threshold;
said depth variation value is negative;
said vertical movement is present;
said first inference indicates that baby is present in said cradle;
said magnitude of said inner movement in said intervention duration exceeds a second threshold; and
said respiratory rate in said intervention duration exceeds a third threshold,
wherein said intervention is identified as corresponding to baby retrieval activity if:
said mass variation value is negative and a magnitude of said mass variation value exceeds said first threshold;
said depth variation value is positive;
said vertical movement is present;
said first inference indicates that baby is absent in said cradle;
said magnitude of said inner movement in said intervention duration is below said second threshold; and
said respiratory rate in said intervention duration is below said third threshold.

10. The method of claim 8, wherein said first camera is an RGB camera,
wherein said an intervention at said cradle is determined to have occurred if boundary movements at said cradle persist for more than a pre-determined threshold interval.

11. A non-transitory machine readable medium storing one or more sequences of instructions for causing a digital processing system to detect presence or absence of a baby in a cradle, wherein said digital processing system is deployed associated with said cradle, wherein execution of said one or more instructions by one or more processors contained in said digital processing system causes performance of the actions of:
generating a first plurality of data records with each data record having an image and a corresponding label indicating whether baby is present or absent in said cradle corresponding to the image;
feeding each image of said first plurality of data records to a first teacher model to cause said first teacher model to infer whether baby is present or absent in said cradle;
checking whether said inferences of said first teacher model match said corresponding labels with a first desired accuracy;
if yes: continuing to use said first teacher model as an operative model to thereafter infer whether baby is present or absent based on corresponding received images;
if no: using a first student model as said operative model thereafter to infer whether baby is present or not based on corresponding received images,
wherein said first student model is formed by knowledge distillation from said teacher model.

12. The non-transitory machine readable medium of claim 11, wherein said generating is performed using an upfront teacher model as said operative model when said digital processing system is deployed upfront upon installation of said cradle,
wherein said fine-tuning said first student model is performed using a first set of training records prior to using said first student model as said operative model, wherein each training record comprises an image and a corresponding label indicating whether baby is present or absent in said cradle corresponding to the image.

13. The non-transitory machine readable medium of claim 12, wherein said first teacher model and said first student model are received after said generating, wherein said first teacher model does not infer with said first desired accuracy,
wherein said first set of training records corresponding to a first subset of said first plurality of data records in performing said fine-tuning of said first student model, wherein said fine-tuning is performed upon said checking determining that said first teacher model does not infer with said first desired accuracy,
said method further comprising:
feeding each image of a second subset of said first plurality of data records to said fine-tuned first student model to cause said fine-tuned first student model to infer whether baby is present or absent in said cradle;
checking whether said inferences of said fine-tuned first student model match said corresponding labels in said second subset with a second desired accuracy;
if yes, using said fine-tuned first student model as said operative model to thereafter infer whether baby is present or absent based on corresponding received images.

14. The non-transitory machine readable medium of claim 13, wherein said operative model is used for inferring in a first duration following said upfront installation, said method further comprising:
generating a second plurality of data records after said first duration, with each data record having an image and a corresponding label indicating whether baby is present or absent in said cradle corresponding to the image;
retrieving a second teacher model and a second student model after said first duration;
feeding each image of said second plurality of data records to said second teacher model to cause said second teacher model to infer whether baby is present or absent in said cradle;
checking whether said inferences of said second teacher model for said second plurality of data records match said corresponding labels with said first desired accuracy;
if yes, using said second teacher model as said operative model to thereafter infer whether baby is present or absent based on corresponding received images.

15. The non-transitory machine readable medium of claim 14, wherein said second teacher model does not infer with said first desired accuracy, said method further comprising:
checking whether said second student model infers with said second desired accuracy;
if no, fine-tuning said second student model with a third subset of said second plurality of data records until said second student model infers with said second desired accuracy; and
using said fine-tuned second student model as said operative model to thereafter infer whether baby is present or absent based on corresponding received images.

16. The non-transitory machine readable medium of claim 15, wherein said first teacher model is fine-tuned at said central server to generate said second teacher model,
wherein said first teacher model is shared by a plurality of said digital processing systems prior to said first duration and said second teacher model is shared by said plurality of digital processing systems after said first duration,
wherein each digital processing system of a plurality of digital processing systems has a respective associated fine-tuned model stored at said central server, wherein said second student model is the corresponding fine-tune model for said digital processing system stored at said central server.

17. The non-transitory machine readable medium of claim 12, wherein said generating comprises:
determining the occurrence of an intervention at said cradle, wherein said intervention is characterized by an intervention-start-time and an intervention-end-time respectively corresponding to the start time instance and end time instance of said intervention, wherein an intervention duration is a time interval bounded by said intervention-start-time and said intervention-end-time;
receiving data from a plurality of sensors associated with said cradle in said intervention duration and prior to said intervention-start-time;
capturing an image of said cradle using a first camera towards said intervention-end-time;
feeding said image to said operative model to cause said operative model to generate a first inference, wherein said first inference indicates whether baby is present or absent in said cradle;
identifying said intervention as corresponding to a baby placement activity or a baby retrieval activity based on said data received from said plurality of sensors and said first inference,
wherein if said intervention is identified as corresponding to a baby placement activity:
recording said intervention-start-time as an end time of a ‘baby absent’ state and said intervention-end-time as a start time of a ‘baby present’ state,
wherein if said intervention is identified as corresponding to a baby retrieval activity:
recording said intervention-start-time as an end time of said ‘baby present’ state and said intervention-end-time as a start time of said ‘baby absent’ state;
labeling a first plurality of images captured using said first camera in a first label-duration bounded by said start time and said end time of said ‘baby present’ state with a label indicating that baby is present in said cradle; and
labeling a second plurality of images captured using said first camera in a second label-duration bounded by said start time and said end time of said ‘baby absent’ state with a label indicating that baby is absent in said cradle,
storing each image of said first plurality of images and said second plurality of images and said respective label as a corresponding data record in a data store on said digital processing system,
wherein said labeling is performed automatically.

18. A digital processing system comprising:
one or more memories to store instructions; and
one or more processors to retrieve said instructions and execute said instructions, wherein execution of said instructions causes said digital processing system to perform the actions of:
generating a first plurality of data records with each data record having an image and a corresponding label indicating whether baby is present or absent in said cradle corresponding to the image;
feeding each image of said first plurality of data records to a first teacher model to cause said first teacher model to infer whether baby is present or absent in said cradle;
checking whether said inferences of said first teacher model match said corresponding labels with a first desired accuracy;
if yes: continuing to use said first teacher model as an operative model to thereafter infer whether baby is present or absent based on corresponding received images;
if no: using a first student model as said operative model thereafter to infer whether baby is present or not based on corresponding received images,
wherein said first student model is formed by knowledge distillation from said teacher model.

19. The digital processing system of claim 18, wherein said generating is performed using an upfront teacher model as said operative model when said digital processing system is deployed upfront upon installation of said cradle,
wherein said fine-tuning said first student model is performed using a first set of training records prior to using said first student model as said operative model, wherein each training record comprises an image and a corresponding label indicating whether baby is present or absent in said cradle corresponding to the image.

20. The digital processing system of claim 19, wherein said first teacher model and said first student model are received after said generating, wherein said first teacher model does not infer with said first desired accuracy,
wherein said first set of training records corresponding to a first subset of said first plurality of data records in performing said fine-tuning of said first student model, wherein said fine-tuning is performed upon said checking determining that said first teacher model does not infer with said first desired accuracy,
said method further comprising:
feeding each image of a second subset of said first plurality of data records to said fine-tuned first student model to cause said fine-tuned first student model to infer whether baby is present or absent in said cradle;
checking whether said inferences of said fine-tuned first student model match said corresponding labels in said second subset with a second desired accuracy;
if yes, using said fine-tuned first student model as said operative model to thereafter infer whether baby is present or absent based on corresponding received images,
wherein said operative model is used for inferring in a first duration following said upfront installation, said method further comprising:
generating a second plurality of data records after said first duration, with each data record having an image and a corresponding label indicating whether baby is present or absent in said cradle corresponding to the image;
retrieving a second teacher model and a second student model after said first duration;
feeding each image of said second plurality of data records to said second teacher model to cause said second teacher model to infer whether baby is present or absent in said cradle;
checking whether said inferences of said second teacher model for said second plurality of data records match said corresponding labels with said first desired accuracy;
if yes, using said second teacher model as said operative model to thereafter infer whether baby is present or absent based on corresponding received images.
